(19) United States
(12) Patent Application Publication
(10) Pub. No.: US 2021/0017556 A1
(43) Pub. Date: Jan. 21, 2021

(54) PROCESS FOR OXIDISING A SUBSTRATE

(71) Applicant: BIOME BIOPLASTICS LIMITED, Southampton, Hampshire (GB)

(72) Inventors: Paul MINES, Southampton, Hampshire (GB); Paul LAW, Southampton, Hampshire (GB); Andrew CARNELL, Southampton, Hampshire (GB); Shane MCKENNA, Southampton, Hampshire (GB); John BLACKER, Southampton, Hampshire (GB); Nikil KAPUR, Southampton, Hampshire (GB)

(21) Appl. No.: 17/040,003

(22) PCT Filed: Mar. 26, 2019
(86) PCT No.: PCT/EP2019/057617

§ 371 Date: Sep. 21, 2020

(30) Foreign Application Priority Data
Mar. 27, 2018 (GB) 1804929.6

(51) Int. Cl. C12P 17/04; C08G 63/181; C12M 1/40; C12M 1/12; C12M 1/00; C12M 1/34; B01F 5/06; B01F 5/04; B01F 13/00; B01F 3/08

(52) U.S. Cl. CPC C12P 17/04; C08G 63/181; C12M 21/18; C12M 23/06; C12M 23/40; C12M 29/14; B01F 2215/0036; B01F 5/0647; B01F 5/0696; B01F 5/0473; B01F 13/0061; B01F 3/0865; C12M 41/32

(57) ABSTRACT

A process for oxidising a substrate selected from hydroxymethylfurfural (HMF), diformylfuran (DFF), hydroxymethylfurancarboxylic acid (HMFCA) and formylfurancarboxylic acid (FFCA). Said process comprises mixing said substrate with catalase, one or more further enzymes and hydrogen peroxide to form a reaction mixture. Said one or more further enzymes have the ability to catalyse oxidation of said substrate. Said hydrogen peroxide is provided at a total molar ratio of at least about 0.1:1 hydrogen peroxide to substrate.

### TECHNICAL FIELD

The present invention relates to a process for oxidising a substrate. The invention relates more particularly, but not necessarily exclusively, to a process for the formation of furandicarboxylic acid and/or intermediate products that are able to be converted to furandicarboxylic acid.

### BACKGROUND

Due to their versatility, polymers, such as plastics, have found wide ranging applications in modern society, and can be found in products ranging from carbonated drinks bottles to mobile phones and surgical equipment. PET (polyethylene terephthalate) is one of the most dominant plastics on the market. The annual worldwide production of PET is approximately 53.3 million tonnes, which makes up 18% of global polymer production. However, as PET is highly stable, it is resistant to biodegradation which poses a significant environmental threat.

PBAT (polybutylene adipate co-terephthalate) is known to be flexible, tough and most importantly biodegradable. PBAT can be blended with other biodegradable polymers and can potentially be used as substitutes for industry standard plastics, such as PET.

Terephthalic acid (TPA) is a precursor used in the production of PET and PBAT. TPA is manufactured by the oxidation of para-xylene, which is derived from petrochemicals. As oil reserves represent a finite source of petrochemicals, there is considerable interest in the development of bio-based plastics derived from biomass, particularly plastics that are biodegradable.

It is known that 5-hydroxymethylfurfural (HMF) can be derived from cellulose via dehydration of glucose and fructose.

Obtaining 2,5-furandicarboxylic acid from 5-hydroxymethylfurfural requires a six-electron oxidation for which numerous metal catalysts and nanoparticles have been employed, such as Au—TiO₂, Au—C modified with Pd, Au-hydrotalcite, Pt—C, Au/TiO₂, and Pt/ZrO₂. However, these reactions require high pressure and/or temperature and additives that decrease the sustainability of the process considerably.

WO2016202858 relates to an alternative process for producing 2,5-furandicarboxylic acid and/or esters thereof via a multistep biocatalytic oxidation reaction of 5-hydroxymethylfurfural (HMF). The multistep biocatalytic oxidation reaction uses, for example, an enzyme selected from xanthine oxidoreductase (XOR), galactose oxidase variant M₃₋₅, aldehyde dehydrogenase, and/or ketoreductase; in conjunction with catalase enzyme.

It is desirable to provide an improved process and/or otherwise to obviate and/or mitigate one or more of the disadvantages with known processes, whether identified herein or otherwise.

### SUMMARY OF THE INVENTION

According to the invention, there is provided a process for oxidising a substrate selected from hydroxymethylfurfural (HMF), diformylfuran (DFF), hydroxymethylfurancarboxylic acid (HMFCA) and formylfurancarboxylic acid (FFCA), said process comprising:

mixing said substrate with catalase, one or more further enzymes and hydrogen peroxide to form a reaction mixture;

wherein said one or more further enzymes have the ability to catalyse oxidation of said substrate and wherein said hydrogen peroxide is provided at a total molar ratio of at least about 0.1:1 hydrogen peroxide to substrate.

There is also provided an apparatus for a flow process of oxidising a substrate, said apparatus comprising:

a primary tube defining a primary fluid passageway for flow of a first fluid (e.g. a liquid);

secondary tubing defining a secondary passageway for adding one or more further fluids [e.g. liquid(s)] to the primary fluid passageway, said secondary tubing having one or more apertures to permit fluid communication of the secondary passageway with the primary passageway.

There is also provided a process for the formation of a mono- or diester of furandicarboxylic acid from furandicarboxylic acid, the process comprising mixing furandicarboxylic acid, an alcohol and a catalyst, wherein the furandicarboxylic acid is obtained by a process for oxidising a substrate as defined above.

There is also provided a process for the formation of a copolymer comprising the copolyester of:
(a) furandicarboxylic acid (FDCA) and/or a mono- or diester of furandicarboxylic acid; and
(b) at least one diol;
wherein the process comprises polymerising components (a) and (b), wherein the furandicarboxylic acid is obtained by a process for oxidising a substrate as defined above, and/or wherein the mono- or diester of furandicarboxylic acid is obtained by a process for the formation of a mono- or diester of furandicarboxylic acid as defined above.

There is also provided a copolymer obtainable by the process for the formation of a copolymer as defined above.

### Definitions

The following definitions apply for terms used herein.

The term “at least one” is synonymous with “one or more”, i.e. one, two, three, four, five, six, or more.

As used herein the term “about” applies to all values and ranges, numeric or otherwise, whether or not explicitly indicated. Those values and ranges generally encompass or refer to a range of values that one skilled in the art would consider equivalent to the recited values (i.e. having the same function or result). Where the term “about” is used in relation to a numerical value, it can represent (in increasing order of preference) a 10%, 5%, 2%, 1% or 0% deviation from that value.

The term “consists essentially of” is used herein to denote that a given copolymer consists of only designated materials and optionally other materials that do not materially affect the characteristic(s) of the claimed copolymer. Suitably, a product which consists essentially of a designated material (or materials) comprises greater than or equal to 85% of the designated material, more suitably greater than or equal to 90%, more suitably greater than or equal to 95%, more suitably greater than or equal to 98%, most suitably greater than or equal to 99% of the designated material(s).

The term “monomer” is one of the art. For the avoidance of any doubt, monomers are molecules that can be bonded to other molecules to form a polymer or a copolymer.

The term “polymer” as used herein may refer to a molecule comprising two or more (such as three or more, four or more, five or more, six or more, seven or more, eight or more, nine or more, or ten or more) monomer units. A polymer may comprise many monomer units, such as 100 or more monomer units.

The term “copolymer” is one of the art. It refers to a polymer comprising two or more different monomer units that are polymerised in a process known as copolymerisation.

Since a copolymer comprises at least two different monomer units, copolymers can be classified based on how the monomer units are arranged to form a polymer chain. Those classifications include “alternating copolymers” (in which the monomers units repeat with a regular alternating pattern), “periodic copolymers” (in which the monomers units are arranged with a repeating sequence), “statistical copolymers” (in which the sequence of monomer units follows a statistical rule), “random copolymers” (in which the monomer units are attached in a random order), and “block copolymers” (in which two or more homopolymer subunits are linked).

The term “complex” includes salts and macrocycles within its definition. Suitable salts include acetate, alkoxide, oxide, sulphate, and halide. The complex may comprise Acac, OEt₃, oxide, sulphate, and phthalocyanine. Exemplary metal complexes include V^{(II)}acac, V^{(V)}OEt₃, V^{(V)}oxide, Vanadyl acac, VO^{(IV)}sulphate, Mn^{(III)}acac, Mn^{(II)}sulphate, Fe(II)phthalocyanine, Fe^{(III)}acac, Fe^{(III)}EDTA, Fe^{(III)}oxide, Co^{(II,III)}ooxide, hematin, and hemin. The complex may include V^{(V)}oxide and VO^{(IV)}sulphate. The complex may include VO^{(IV)}sulphate. Those skilled in the art will appreciate that acac is acetylacetone.

The term “immobilised periplasmic aldehyde oxidase (PaoABC)” and “immobilised catalase” refers to periplasmic aldehyde oxidase or catalase that is attached to, or entrapped in, an inert, insoluble material. Materials suitable for immobilising enzymes, such as PaoABC and catalase, are known in the art and include epoxide-based resins, such as Eupergit CM; hydrogels, such as those comprising polyvinyl imidazole (PVI), polyethylene imine (PEI), and/or polyethylene glycol (PEG) stabilisers; enzyme affinity columns, such as those used in immobilised metal ion affinity chromatography (IMAC); and as cross-linked enzyme aggregates (CLEA).

The term “biodegradable” as used herein, means degradable by means of microorganisms, such as fungi, bacteria, viruses, algae, etc., and/or by exposure to enzymatic mechanisms. As applied to a given product, such as a polymer/copolymer, the requirement “biodegradable” should be understood to be met if the majority of that product is biodegradable, i.e. if the product is “partially” biodegradable. It is not intended that the entire product must be biodegradable. Suitably, at least 60% of the product may be biodegradable, on a weight basis; optionally at least 70%; optionally at least 80%; optionally at least 90%; optionally at least 95%; optionally 100% of the product may be biodegradable. Generally speaking, greater biodegradability is preferred.

The term “compostable” means degradable to form compost. As applied to a given product, such as a polymer/copolymer, the requirement “compostable” should be understood to be met if the majority of that product is compostable, i.e. if the product is “partially” compostable. It is not intended that the entire product must be compostable. Suitably, at least 60% of the product may be compostable, on a weight basis; optionally at least 70%; optionally at least 80%; optionally at least 90%; optionally at least 95%; optionally 100% of the product may be compostable. Generally speaking, greater compostability is preferred.

The term “glass transition temperature”, as applied to a component comprising a polymer/copolymer (such as a blend) should be understood to denote the relevant transition temperature of the predominant polymer in the blend (i.e. major component on a weight basis). In instances where polymers/copolymers in a blend are fully dispersible (e.g. miscible) in one another, then the glass transition of the blend may comprise properties combined from each of the polymers/copolymers.

The terms “increased pressure” and “reduced pressure” are ones of the art and includes all pressure that are, respectively, above or below atmospheric (or ambient) pressure (e.g. about 95 to 105 kPa, such as about 100 kPa). Similarly, “increased temperature” and “reduced temperature” includes all temperatures that are, respectively, above or below ambient temperature (e.g. room temperature, about 23 to 25.5° C.).

The term “alkyl” refers to a saturated (no double or triple bonds) aliphatic hydrocarbon radical, including straight-chain, and, where possible, branched-chain and cyclic groups. Where the alkyl group refers to a range, such as C₁ to C₆, it is to be understood that it includes each member of the range, i.e. C₁, C₂, C₃, C₄, C₅, and/or C₆.

The term “alkenyl” refers to an alkyl radical that contains in the straight or branched hydrocarbon chain and one or more double bonds. Examples of alkenyl groups include allenyl, vinylmethyl and ethenyl.

The term “alkynyl” refers to an alkyl group that contains in the straight or branched hydrocarbon chain one or more triple bonds. Examples of alkynyls include ethynyl and propynyl.

Alkyl, alkenyl and alkynyl groups are optionally “C₂₋₂₀ alkyl”, “C₂₋₂₀ alkenyl” and “C₂₋₂₀alkynyl”, optionally “C₂₋₁₅ alkyl”, “C₂₋₁₅ alkenyl” and “C₂₋₁₅ alkynyl”, optionally “C₂₋₁₂ alkyl”, “C₂₋₁₂ alkenyl” and “C₂₋₁₂ alkynyl”, optionally “C₂₋₁₀ alkyl”, “C₂₋₁₀alkenyl” and “C₂₋₁₀ alkynyl”, optionally “C₂₋₈ alkyl”, “C₂₋₈ alkenyl” and “C₂₋₈ alkynyl”, optionally “C₂₋₆ alkyl”, “C₂₋₆ alkenyl” and “C₂₋₆ alkynyl” groups, respectively.

A heteroalkyl group is an alkyl group as described above, which additionally contains one or more heteroatoms. Heteroatoms are optionally selected from O, S, N, P and Si. A heteroaliphatic group is an aliphatic group as described above, which additionally contains one or more heteroatoms.

An aryl group is a monocyclic or polycyclic ring system having from 5 to 20 carbon atoms. An aryl group is optionally a “C₆₋₁₂ aryl group” and is an aryl group constituted by 6, 7, 8, 9, 10, 11 or 12 carbon atoms and includes condensed ring groups such as monocyclic ring group, or bicyclic ring group and the like.

A heteroaryl group is an aryl group having, in addition to carbon atoms, from one to four ring heteroatoms which are optionally selected from O, S, N, P and Si.

The term “diol” refers to a compound of formula

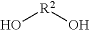

wherein R² is a straight or, where possible, branched or cyclic C₂ to C₁₀ saturated alkylene, optionally a C₂ to C₆ saturated alkylene, and optionally C₂ to C₄ saturated alkylene, or a mixture thereof.

The skilled person will understand that for a diol to be branched or cyclic then at least three carbon units are required. Non-limiting examples of branched diols useful in the invention are 1,2-propanediol, 1,2-butanediol, 2,2-dimethyl-1,3-propanediol. Non-limiting examples of cyclic diols useful in the invention are 1,2-cyclopentanediol, 1,2-cyclobutanediol, and 1,2-cyclopentanediol. The diols may also be branched, cyclic diols, e.g. 3-methyl-1,2-cyclopropanediol.

The term “aliphatic dicarboxylic acid or a mono- or diester derivative thereof” refers to a compound of formula

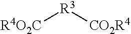

wherein R³ is a straight or, where possible, branched or cyclic, C₁ saturated or C₂ to C₁₀ saturated or unsaturated alkylene, optionally C₂ to C₆ saturated or unsaturated alkylene, and optionally C₂ to C₄ saturated or unsaturated alkylene, or combinations thereof, and wherein each R⁴ independently represents H or a straight, or where possible branched or cyclic, C₁ to C₆ alkyl group, such as a C₁ to C₄ alkyl group, optionally H or a C₁ or C₂ alkyl group. Optionally the two R⁴ groups are the same.

The term “halide” or “halogen” are used interchangeably and, as used herein mean an ion derived from IUPAC group number 17 of the periodic table. The halide/halogen may be fluoride, a chloride, a bromide, an iodide and the like.

An alkoxy group is optionally a “C₁₋₂₀ alkoxy group”, optionally a “C₁₋₁₅ alkoxy group”, optionally a “C₁₋₁₂ alkoxy group”, optionally a “C₁₋₁₀ alkoxy group”, optionally a “C₁₋₈ alkoxy group”, optionally a “C₁₋₆ alkoxy group”.

Sulphate is the dianion of SO₄.

### DETAILED DESCRIPTION

According to the invention, there is provided a process for oxidising a substrate selected from hydroxymethylfurfural (HMF), diformylfuran (DFF), hydroxymethylfurancarboxylic acid (HMFCA) and formylfurancarboxylic acid (FFCA), said process comprising:
mixing said substrate with catalase, one or more further enzymes and hydrogen peroxide to form a reaction mixture;
wherein said one or more further enzymes have the ability to catalyse oxidation of said substrate and wherein said hydrogen peroxide is provided at a total molar ratio of at least about 0.1:1 hydrogen peroxide to substrate.

The present invention relates to a process for production of furandicarboxylic acid (FDCA) and/or intermediate products that are able to be converted to furandicarboxylic acid (FDCA).

Previous work in enzymatic production of furandicarboxylic acid (FDCA) from hydroxymethylfurfural (HMF) employed catalase enzyme to remove hydrogen peroxide from the system, which is produced as a by-product from certain enzymatic processes. Hydrogen peroxide degrades and thereby generates reactive oxidising species capable of attacking biological molecules. This may reduce enzyme activity over time and thus negatively impact the production method as described above.

Addition of catalase to the system degrades hydrogen peroxide into species which do not attack biological molecules in the same way, thus preserving enzymatic activity.

The present invention is based, in part, on the finding that actively adding hydrogen peroxide to the mixture (i.e. adding above by-product levels naturally produced during the enzymatic processes) promotes oxidation of the substrates discussed herein, improving both the rate and yield of oxidation.

Without wishing to be bound by theory, it is thought that a limiting factor in the progress of enzymatic-catalysed oxidation reactions is in liquid/gas mass transfer of oxidant species (such as oxygen). This is thought to be due to the poor solubility of oxygen in water (typically used as a medium for enzymatic processes). As used in the present invention, catalase converts hydrogen peroxide into oxygen, thus providing a source of oxygen in situ that may be used as the oxidant in the reaction. This enables the water medium to become highly saturated with oxygen, thereby addressing the limiting factor noted above.

Moreover, since oxygen is provided in situ, it may be possible to reduce or eliminate the need to add further oxidant species (e.g. oxygen) to the reaction mixture, which is often otherwise provided using highly pressurised systems. This reduces the risk of explosion/fire in the process. Additionally, provision of oxidant species, such as oxygen, is typically done by bubbling gas through the reaction mixture (e.g. by sparging). Such bubbling can, however, damage components of the reaction (e.g. the enzymes).

Still further, hydrogen peroxide is broken down in situ into reactive oxygen species which may further enhance oxidation of the substrates.

The total molar ratio may be understood to refer to the ratio of the total amount of hydrogen peroxide to the total amount of substrate used in the process.

Hydrogen peroxide may be provided at a total molar ratio of at least about 1:1 hydrogen peroxide to substrate, optionally at least about 2:1, optionally at least about 3:1 hydrogen peroxide to substrate. Hydrogen peroxide may be provided at a total molar ratio of up to about 20:1 hydrogen peroxide to substrate, optionally about 15:1 hydrogen peroxide to substrate, optionally up to about 10:1 hydrogen peroxide to substrate.

Mixing may comprise mixing said substrate with catalase and one or more further enzymes to form a mixture, the process comprising adding hydrogen peroxide to the mixture to form the reaction mixture.

Mixing may comprise mixing said substrate with catalase and one or more further enzymes to form a mixture, the process comprising providing a flow of said mixture and adding hydrogen peroxide to the mixture flow to form the reaction mixture. Such processes are described herein as flow processes.

Flow processes may be considered as continuous processes. Continuous processes may permit improvements in the rates of reaction and/or productivity (e.g. as compared to equivalent batch processes). Without wishing to be bound by theory, it is believed that the residence time in continuous processes (such as flow processes) gives such improvements in productivity. In an implementation, the flow process of the invention has been found to offer a 5-fold improvement in comparative batch processes.

A flow process may be useful for admitting a relatively low local concentration of hydrogen peroxide into the reaction mixture, while providing a sufficient amount of hydrogen peroxide on a bulk basis. This is particularly true for processes employing multiple streams of hydrogen peroxide. Such admission may be useful for overcoming rate limiting issues with liquid/gas mass transfer of oxidant species (such as oxygen). Additionally, such addition may be useful to obviate or mitigate oxidative damage as may otherwise be caused by high local peroxide concentration (and its degradation to reactive oxygen species).

The “total molar ratio” in continuous flow processes may refer to the total amount of hydrogen peroxide to the total amount of substrate for a given volume of reaction mixture flowing over a given period of time. By way of example, if a continuous process involves flow of 1 L reaction mixture per hour, then the “total molar ratio” may be considered to refer to the total amount of hydrogen peroxide to the total amount of substrate employed in that 1 L of reaction mixture over that hour. In such processes, the “total molar ratio” may be understood to refer to steady-state operation.

Said flow may follow a tortuous passageway having one or more bends. Said hydrogen peroxide may be added at the bend, a subset of said bends or each bend.

The primary tube may be provided with one or more flow disruptors, such as particles. These may be useful to improve mixing in the apparatus. The flow disruptors may be inert and thereby substantially unreactive with respect to the reagents and/or solvents employed during the reaction. The flow disruptors may be glass particles, such as glass beads, or may be Raschig rings, super rings, Pall rings, or the like.

The flow disruptor(s) may be sized to closely fit inside the primary tube, e.g. having a particle diameter (such as an average particle diameter) of at least about 0.1% of the diameter of the primary tube, such as at least about 0.5%, such as at least about 1%, such as at least about 5%, such as at least about 10%, such as at least about 20%. The flow disruptor(s) may have a particle diameter (such as an average particle diameter) of at most about 35% of the diameter of the primary tube, such as at most about 30%, such as at most about 25%. The flow disruptor(s) particle diameter (such as an average particle diameter) may be between about 0.1% and about 35% of the diameter of the primary tube, such as between about 0.1% and about 30%, such as between about 0.1% and about 25%.

The flow disruptor(s) may be substantially stationary within the primary tube (e.g. substantially stationary with respect to the direction of fluid flow).

Adding hydrogen peroxide may comprise adding hydrogen peroxide at an amount sufficient to bring the molar ratio of the hydrogen peroxide in the reaction mixture to within about 50% of a predetermined molar ratio, optionally within about 40%, optionally within about 30%, optionally within about 20%, optionally within about 10%, optionally within about 5%, optionally within about 5% of said predetermined molar ratio, optionally approximately to said predetermined ratio.

The predetermined ratio may be equal to the total molar ratio, or may be less than the total molar ratio. It will be appreciated that hydrogen peroxide may be added in the process more than once. In such instances, the predetermined ratio may be a fraction of the total molar ratio, the fraction being calculated by dividing the total molar ratio by the number of additions of hydrogen peroxide. By way of example, if there are three additions of hydrogen peroxide, then the predetermined molar ratio may be one third of the total molar ratio. If there is only a single addition of hydrogen peroxide, then the predetermined ratio may be equal to the total molar ratio.

Adding may comprise adding one or more streams of hydrogen peroxide, such as by pumping hydrogen peroxide. Optionally, said pumping may be conducted through one or more capillary tubes defining fluid passageways for admission of said hydrogen peroxide. Hydrogen peroxide may be added by means of one or more syringe pumps.

Adding hydrogen peroxide may comprise adding multiple individual streams of hydrogen peroxide. Adding may comprise adding at least 5 individual streams of hydrogen peroxide, optionally at least 7, optionally at least 10, optionally at least 11 individual streams of said hydrogen peroxide during the process.

In general, the process of the invention may be conducted on any scale. On a laboratory scale, adding may comprise adding 5 to 20 individual streams of hydrogen peroxide, optionally 7 to 15, optionally 10 to 12, optionally 11 individual streams of said hydrogen peroxide during the process. It will be appreciated that a large-scale process may employ a larger number of individual streams of hydrogen peroxide.

In a flow process, streams may be provided at different parts of the fluid flow path, e.g. at evenly distributed points along a flow path. By way of example, if 11 streams are provided along a 10 cm long flow path, then a stream may be provided 0 cm along the flow path (e.g. at an inlet end of a tube), with additional streams being provided at 1 cm intervals.

Adding said hydrogen peroxide may comprise continuously adding or periodic adding.

In terms of continuous mode of addition, hydrogen peroxide may be understood to be continually or constantly added during the course of a reaction. This may be achieved by, for example, adding a stream or flow of hydrogen peroxide with either a constant flow rate or with a variable flow rate. In other words, the hydrogen peroxide is added in an essentially non-stop fashion. It is noted, however, that non-stop addition of hydrogen peroxide may need to be briefly interrupted for practical considerations, for example to refill or replace a container of hydrogen peroxide from which it is being added.

Periodic adding may comprise adding 1, 2, 3, 4, 5, 6, 7, 8, 9, or 10 (or more) portions of said hydrogen peroxide. Periodic adding may comprise adding two or more portions of hydrogen peroxide, the addition of each portion being separated by a time interval of from about 1 second to about 60 minutes, optionally about 5 minutes to about 25 minutes, optionally about 10 minutes to about 20 minutes, optionally about 15 minutes. By way of example, a portion of hydrogen peroxide may be added at the outset (i.e. at the beginning of reaction), to provide an initial dose of hydrogen peroxide. One or more further portions may thereafter be added, such as 15 minutes thereafter, 30 minutes thereafter, 45 minutes thereafter, etc.

Said flow has a velocity and the process may be configured to maintain a predetermined flow velocity (e.g. constant velocity) along a path of the flow (e.g. from inlet to outlet). Such configuration may be achieved, for example, by varying the cross-sectional area of a tube through which the reaction mixture flows.

Said provision of hydrogen peroxide may be configured to maintain the predetermined flow velocity (for example by adjusting the cross-sectional area of tubes through which said hydrogen peroxide is admitted).

Configuration in these ways may be useful so that admitted reagents have sufficient residency time to undergo substantial reaction.

The molar ratio of each component (e.g. the substrate and/or hydrogen peroxide) may be determined in situ, e.g. by direct measurement of the reaction mixture. Such a measurement may comprise taking an aliquot sample of the reaction mixture at a given timepoint and performing analysis by reverse-phase high pressure liquid chromatography.

Alternatively, the molar ratio of each component at a given timepoint may be estimated. By way of example, the molar ratio may be estimated based on knowledge of the rate of reaction, the molar ratios of each component mixed and the elapsed reaction time (to a given time point).

Said one or more further enzymes may comprise metal. Said one or more further enzymes may comprise iron, molybdenum and/or copper metal. Such metals catalyse the breakdown of hydrogen peroxide into to reactive hydroxyl and hydroperoxyl radicals capable of attacking biological molecules. This may reduce enzyme activity over time and thus negatively impact the production method.

In the context of the invention, an enzyme comprising metal may be understood to refer to the catalytic site comprising said metal. For example, the catalytic site may comprise a ligand coordinating said metal. The ligand may be a free radical ligand.

Hydroxymethylfurfural (HMF) can undergo a series of oxidation steps to produce furandicarboxylic acid (FDCA). First, hydroxymethylfurfural (HMF) can be oxidised to diformylfuran (DFF) by oxidation of the alcohol moiety thereon to an aldehyde. Alternatively, hydroxymethylfurfural (HMF) can be oxidised to hydroxymethylfurancarboxylic acid (HMFCA) by oxidation of the aldehyde moiety thereon to a carboxylic acid. Diformylfuran (DFF) can in turn be oxidised to 5-formylfuran-2-carboxylic acid (FFCA) by oxidation of one of the aldehyde moieties thereon to a carboxylic acid. Diformylfuran (DFF) can also be oxidised to formylfurancarboxylic acid (FFCA) by oxidation of the alcohol moiety thereon to an aldehyde. Finally, formylfurancarboxylic acid (FFCA) can be oxidised to the final product formylfurancarboxylic acid (FFCA) by oxidation of the aldehyde moiety thereon to a carboxylic acid.

The substrate may be selected from 5-(hydroxymethyl)-2-furfuraldehyde (HMF), 2,5-diformylfuran (DFF), 5-hydroxymethyl-2-furancarboxylic acid (HMFCA), 5-formylfuran-2-carboxylic acid (FFCA) and 2,5-furandicarboxylic acid (2,5-FDCA). The process may be for the formation of 2,5-furandicarboxylic acid (2,5-FDCA). An exemplary reaction scheme is as follows:

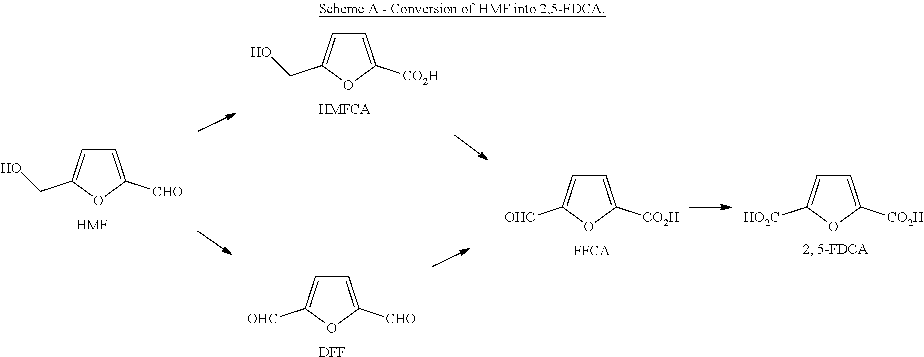

Said one or more further enzymes may comprise aldehyde oxidase, aldehyde dehydrogenase (ALD), alcohol oxidase, galactose oxidase variant (such as galactose oxidase variant M₃₋₅, GOase M₃₋₅), ketoreductase (KRED) and/or nicotinamide oxidase (NOX).

Such enzymes have utility in one or more of the oxidation steps of the present invention. By way of example, xanthine oxidoreductase (XOR) may have utility in the oxidation of aldehyde moieties to carboxylic acids and galactose oxidase variant M₃₋₅ (GOase M₃₋₅) may have utility in the oxidation of alcohols to aldehydes, as follows:

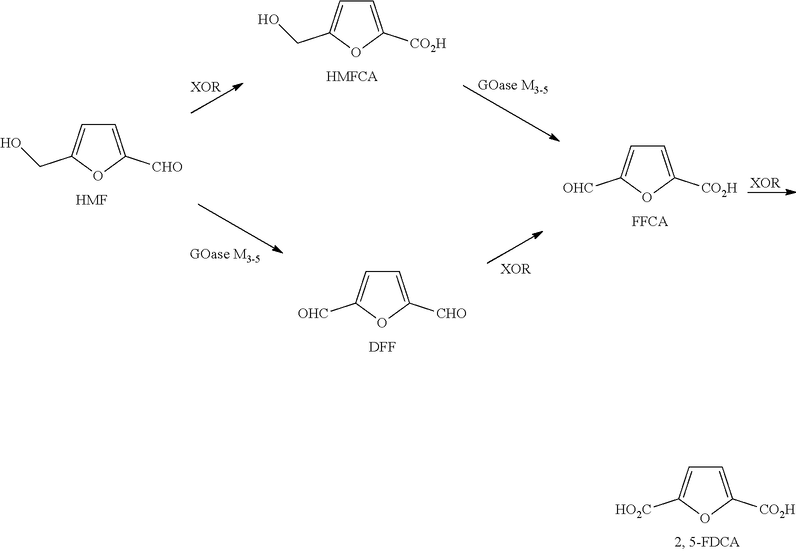

Such steps can be conducted separately (e.g. involving purification of intermediate products after each step), or can be conducted sequentially (e.g. in a one-pot manner) to effect complete oxidation of hydroxymethylfurfural (HMF) to furandicarboxylic acid (FDCA).

Other combinations of enzymes and/or individual enzymes may be useful for effecting other individual steps or series of steps in the production of 2,5-furandicarboxylic acid (FDCA). By way of example, aldehyde dehydrogenase (ALD) and nicotinamide oxidase (NOX) may act in tandem to provide a one-pot oxidation route from diformylfuran (DFF) to furandicarboxylic acid (FDCA), as follows:

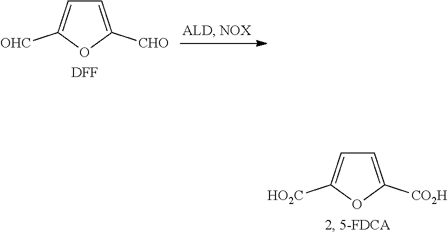

Ketoreductase (KRED) may be useful for providing an all-in-one (i.e. one-pot) oxidation route from hydroxymethylfurfural (HMF) to furandicarboxylic acid (FDCA), as follows:

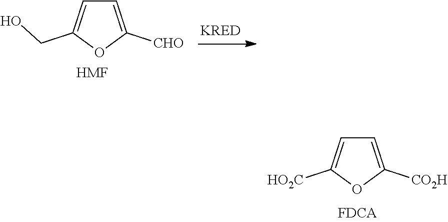

Suitable xanthine oxidoreductases include E. coli XDH, Rhodococcus capsulatus xanthine dehydrogenase (XDH) single variant E232V, and double mutant XDH E232 V/R310, and periplasmic aldehyde oxidase (PaoABC). The xanthine oxidoreductase may be periplasmic aldehyde oxidase (PaoABC).

Periplasmic aldehyde oxidase can oxidise hydroxymethylfurfural, diformyl furan and formylfurancarboxylic acid, and is therefore particularly useful.

The aldehyde dehydrogenase may comprise aldehyde dehydrogenase 3 (ALD-003). Suitably, aldehyde dehydrogenase 3 is available from Prozomix Limited, Station Court, Haltwhistle, Northumberland, NE49 9HN, UK (catalogue name: Pro-ALDH (003)).

Suitable ketoreductases include ketoreductase 20 (KRED-020), KRED-089, KRED-141, KRED-143, KRED-163, and KRED-190; particularly KRED-089. These KREDs are available from Prozomix Limited, Station Court, Haltwhistle, Northumberland, NE49 9HN, UK, as an Aldo-Keto Reductase Panel (product name kREDy-to-go (AKR/ADH; kit of 96 enzymes); catalogue no. PRO-AKRP(MTP)).

Catalase may be provided in a mass ratio of from about 1:100 to about 1:1, such as from 1:75 to about 1:20, for instance from about 1:50 to about 1:30 with respect to the substrate [e.g. hydroxymethylfurfural (HMF)]. Additional catalase can be added to the reaction as required.

An exogenous electron acceptor, such as DCPIP (2,6-dichloro-4-[(4-hydroxyphenyl)imino]cyclohexa-2,5-dien-1-one) can be used increase the rate of oxidation.

Said one or more further enzymes may comprise (a) galactose oxidase variant M₃₋₅ (GOase M₃₋₅) and/or ketoreductase (KRED); and (b) one or more of xanthine oxidoreductase (XOR), aldehyde dehydrogenase (ALD) and nicotinamide oxidase (NOX).

In the process of the invention, (a) and (b) can be mixed simultaneously or sequentially. In case of sequential mixing, the point at which (b) is mixed after (a) can be contingent on the conversion of the relevant substrate by (a). Optionally at least about 50%, such as at least about 70%, optionally at least about 80%, optionally at least about 90%, and optionally about 100% of the relevant substrate may be converted by (a) and before (b) is mixed.

The amount of a given chemical species (e.g. 5-hydroxymethylfurfural, diformylfuran, etc.) present in the process can be determined by reverse-phase high pressure liquid chromatography (RP-HPLC).

Further oxidants, such as air, may be added during the process of the present invention. Suitable oxidants include oxygen (O₂) and DCPIP (2,6-dichloro-4-[(4-hydroxyphenyl)imino]cyclohexa-2,5-dien-1-one).

It is advantageous to use sufficient buffer capacity to control the pH of the reaction and to drive the reaction to completion. For instance, the pH for the conversion of diformyl furan (DFF) to furandicarboxylic acid (FDCA) using periplasmic aldehyde oxidase (PaoABC) at substrate concentrations of about 100 Nm, can drop (e.g. to less than 5, which is below the optimum pH of between 6 to 8). Any suitable buffer can be used in the reaction. Particularly useful buffers are phosphate buffers, such as a potassium phosphate buffer.

Thus, the processes of the present invention are optionally carried out in a buffered reaction mixture with a pH of 6 to 8. Optionally, the reaction mixture is buffered with a phosphate buffer, optionally potassium phosphate buffer. The pH may be determined by any known means. The pH may be determined using a pH meter and a probe.

The pH can be controlled in other ways, such as by direct addition of alkaline species to the reaction mixture. Suitable alkaline species include alkali metal hydroxides (e.g. lithium, sodium and/or potassium hydroxide, optionally sodium hydroxide and/or potassium hydroxide, optionally sodium hydroxide). Addition of alkaline species may be conducted in a metered fashion, to adjust the amount of alkaline species added. The amount of alkaline species added may be adjustable depending on the pH of the solution, such as a pH determined as set out in the preceding paragraph.

The substrate (e.g. hydroxymethylfurfural) may be provided in solution for the purposes of the reaction, optionally an aqueous solution. Optionally, hydroxymethylfurfural may be provided in a solution at a concentration of from about 1 mM to about 1,000 mM, such as from about 10 mM to about 750 mM, for instance from about 50 mM to about 500 mM.

The process of the present invention can be conducted at any suitable temperature. Those skilled in the art will understand that a suitable temperature for enzymatic processes may be from 0° C. to about 60° C., such as from about 20° C. to about 50° C., for instance from about 30° C. to about 40° C. The processes of the present invention may be carried out at about 37° C. The temperature may be maintained by any suitable means, for instance using a shaking incubator. This has the advantage of agitating the reaction mixture whilst maintaining the suitable temperature.

Thermostable enzymes may be employed in the processes of the invention. Thus, a suitable temperature for thermostable enzymatic processes may be from about 0° C. to about 120° C., such as from about 20° C. to about 120° C., for instance from about 30° C. to about 120° C., such as from about 40° C. to about 120° C., for instance from about 60° C. to about 100° C. The temperature may be maintained by any suitable means, for instance using a shaking incubator. This has the advantage of agitating the reaction mixture whilst maintaining the suitable temperature.

Substrates, such as furandicarboxylic acid (FDCA), can be isolated by a range of means, for instance by heat treatment of the reaction solution to precipitate the enzyme, followed by centrifugation, acidification and filtration.

The enzymes of the present invention can be produced in a purified or partially purified form or as a component of a cell lysate. Alternatively, the process can be catalysed by using a suitable naturally occurring or modified bacterium which includes the required biocatalysts.

Said mixing may further comprise mixing horseradish peroxidase (HRP) and/or metal complex.

The horseradish peroxidase may be mixed in an amount of from about 1 to about 400 mol %, such as from about 5 to about 100 mol %, optionally from about 10 to about 50 mol %, based on the amount of substrate (e.g. hydroxymethylfurfural, HMF).

Preferred metal complexes include those comprising a transition metal, such as vanadium, manganese, iron and cobalt. The metal may be in any oxidation state, such as (I), (II), (III), (IV), and (V). Any suitable complex of the metal may be used.

The metal complexes may be mixed in an amount of from about 1 to about 400 mol %, such as from about 5 to about 100 mol %, optionally from about 10 to about 50 mol %, based on the amount of substrate (e.g. hydroxymethylfurfural, HMF).

Said mixing may further comprise mixing nicotinamide adenine dinucleotide phosphate (NADP⁺) and/or nicotinamide adenine dinucleotide (NAD⁺).

Said one or more further enzymes may comprise nicotinamide oxidase (NOX) and said mixing may comprise mixing nicotinamide adenine dinucleotide phosphate (NADP⁺) and/or nicotinamide adenine dinucleotide (NAD⁺).

Said one or more further enzymes may comprise ketoreductase (KRED) and said mixing may comprise mixing nicotinamide adenine dinucleotide phosphate (NADP⁺) and/or nicotinamide adenine dinucleotide (NAD⁺).

Said mixing may comprise mixing both aldehyde dehydrogenase (ALD) and nicotinamide oxidase (NOX). Nicotinamide oxidase may be mixed in an amount of from about 50 weight % to about 180 weight %, such as from about 60 to about 150 weight %, optionally from about 65 weight % to about 140 weight %, based upon the amount of aldehyde dehydrogenase (ALD).

NADP⁺ or NAD⁺ may be mixed in an amount of from about 5 mol % to about 50 mol %, such as from about 10 mol % to about 30 mol %, optionally about 20 mol %, based upon the amount of substrate (e.g. hydroxymethylfurfural, HMF).

When NADP⁺ is present in the process, it may be used in conjunction with nicotinamide oxidase (NOX), optionally wherein the nicotinamide oxidase (NOX) is nicotinamide oxidase 1 (NOX-1). When NAD⁺ is present in the reaction, it may be used in conjunction with nicotinamide oxidase (NOX), optionally wherein the NOX is nicotinamide oxidase 9 (NOX-9).

Said catalase may be immobilised. Immobilised catalase, such as CLEA catalase, may be provided in a mass ratio of from about 1:100 to about 1:1, such as from 1:75 to about 1:20, for instance from about 1:50 to about 1:30, based upon the amount of catalase to the amount of substrate (e.g. hydroxymethylfurfural, HMF). Additional immobilised catalase can be added to the reaction as required.

Said one or more further enzymes may comprise one or more immobilised enzymes. Said one or more further enzymes may comprise immobilised periplasmic aldehyde oxidase (PaoABC) and/or immobilised galactose oxidase variant [such as galactose oxidase variant M₃₋₅ (GOase M₃₋₅)].

Said diformylfuran (DFF), hydroxymethylfurancarboxylic acid (HMFCA) and formylfurancarboxylic acid (FFCA) may be obtained by oxidation of hydroxymethylfurfural (HMF).

For the purpose of this invention, the hydroxymethylfurfural (HMF) may be formed from glucose and fructose. The glucose and fructose is optionally obtained from lignocelluloses. Such transformations are well known to those skilled in the art, and is summarised as follows:

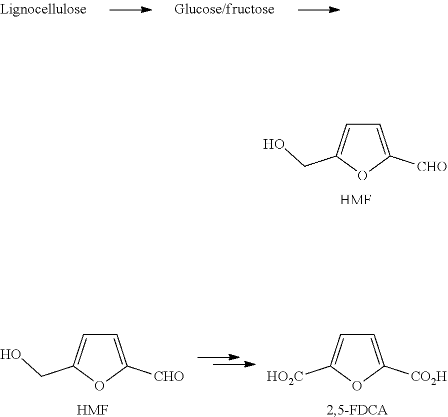

The present invention therefore provides a novel biocatalytic route to furandicarboxylic acid (FDCA) from renewable feedstocks, such as lignocellulose, highlighting the opportunity for bioconversion of lignocellulose into aromatic products using biotechnology.

Since lignocellulose is an abundant, inexpensive and sustainable resource, it may be possible to combine this technology with existing industrial processes in order to generate value-added products from lignocellulose streams.

According to the invention, there is also provided an apparatus for a flow process of oxidising a substrate, said apparatus comprising:
a primary tube defining a primary fluid passageway for flow of a first fluid (e.g. a liquid);
secondary tubing defining a secondary passageway for adding one or more further fluids [e.g. liquid(s)] to the primary fluid passageway, said secondary tubing having one or more apertures to permit fluid communication of the secondary passageway with the primary passageway.

The total cross-sectional area of the aperture(s) of the secondary tubing may be about 10 to 30 times smaller than the cross-sectional area of the primary passageway, optionally about 15 to 25 times smaller, optionally about 20 times smaller than the cross-sectional area of the primary passageway.

The apparatus may be configured to control flow of fluid through said primary passageway and/or flow of fluid through said secondary passageway based on a molar ratio of components in the first fluid of the primary passageway. By way of example, the apparatus may be configured to increase the flow of fluid through said secondary passageway upon measuring a relatively low molar ratio (e.g. below a predetermined molar ratio) of hydrogen peroxide to substrate in said primary passageway. Alternatively, or additionally, the apparatus may be configured to decrease the flow of fluid through said primary passageway upon measuring a relatively low molar ratio (e.g. below a predetermined molar ratio) of hydrogen peroxide to substrate in said primary passageway.

The primary tube may be configured to provide a tortuous passageway, having one or more bends. The or each aperture of the secondary tubing may be provided at the bend, a subset of said bends or each bend in the primary passageway.

In the event that the primary tube does not comprise bends, the apertures may be provided at points along a periphery of the primary tube (i.e. in the tube walls).

The secondary tubing may comprise at least 5 individual fluid passageways, optionally at least 7, optionally at least 10, optionally at least 11 individual fluid passageways. The secondary tubing may comprise 5 to 20 individual fluid passageways, optionally 7 to 15, optionally 10 to 12, optionally 11 individual fluid passageways.

The secondary tubing may comprise a main secondary tube in fluid communication with a series of further secondary tubes, said secondary tubes each having the said apertures for fluid communication with the primary passageway, said further secondary tubes fluidly connecting the primary passageway and the fluid passageway of the main secondary tube.

The secondary tubing may be configured to admit hydrogen peroxide.

The primary tube may comprise an inlet end. The inlet end may be for admission of components into the fluid passageway of the primary tube. The inlet end of the primary tube may be connectable to one or more additional fluid streams for supply of one or more components for reaction in the apparatus. The primary tube may comprise an outlet end (e.g. an opening downstream of the inlet end). The apparatus may comprise a circulator (e.g. a pump) for circulating fluids leaving the primary tube outlet back towards the inlet end.

The primary tube may comprise a tortuous fluid passageway (i.e. including one or more bends). The outlet aperture, a subset of said outlet apertures or each outlet aperture of said one or more individual fluid passageways of the secondary tubing may be provided at said bend(s). It will be appreciated that if there is only one such fluid passageway of the secondary tubing, its outlet aperture may be provided at one said bend. However, if there are multiple such fluid passageways of the secondary tubing (each comprising an outlet aperture), these may be configured such that each of said outlet apertures is provided at a respective bend. Multiple outlet apertures of the secondary tubing may be provided at a single bend.

It is to be understood that said secondary tubing may be configured to admit one component (only, such as hydrogen peroxide) or a mixture of components at the bend, a subset of said bends or each bend.

Said secondary tubing may comprise one or more main secondary tubes, the or each secondary tube defining an individual fluid passageway (e.g. for flow of said hydrogen peroxide) and being in fluid communication with a series of further subordinate secondary tubes, said subordinate secondary tubes each having the said apertures for fluid communication with the primary passageway, said further subordinate secondary tubes fluidly connecting the primary passageway and the fluid passageway of the main secondary tube. In operation, said one or more further components may flow through said main secondary tube into the primary tube through the series of subordinate secondary tubes.

The cross-sectional area and/or length of tubes in the secondary tubing may be configured to maintain a predetermined (e.g. constant) flow velocity along the length of the primary tube.

The cross-sectional area of the primary tube may be configured to maintain a predetermined (e.g. constant) flow velocity along the length of the primary tube.

Configuration in these ways may be useful so that reagents admitted into the primary tube via the secondary tubing have sufficient residency time in the apparatus to undergo substantial reaction.

A reaction may be completed upon full traversal of the fluid through a path defined by the primary tube (i.e. the reaction may be complete in fluids exiting the primary tube). Thus, products may be collected from an outlet end of the tube (e.g. an opening downstream of the inlet end), with components being continuously added at the inlet end. Suitably, products collected from the tubing and components admitted into the tubing may be collected and/or admitted by syringes.

A reaction may require multiple passes through the primary tube to reach completion. In such an implementation, the apparatus may employ a circulator to circulate fluids leaving the primary tube outlet back towards the inlet end. Circulation may be conducted any number of times, depending on the extent of reaction.

The apparatus may further comprise tertiary and optionally quaternary tubing defining tertiary and optionally quaternary passageways, each of said tertiary and optionally quaternary tubing being configured to admit fluid (e.g. liquid) into the primary fluid passageway, said tertiary/quaternary tubing having one or more apertures to permit fluid communication of the tertiary/quaternary passageway with the primary passageway. Said tertiary/quaternary tubing may be configured to admit different fluids than the secondary tubing.

Optional features of the secondary tubing outlined above equally provide optional features for the tertiary/quaternary tubing as set out above, mutatis mutandis. By way of example, the total cross-sectional area of the aperture(s) of each of the tertiary/quaternary tubing may be about 10 to 30 times smaller than the cross-sectional area of the primary passageway; the apparatus may be configured to control flow through the tertiary/quaternary passageways; the tertiary/quaternary tubing may comprise at least 5 individual fluid passageways; etc.

The apparatus may further comprise one or more outlets. The or each outlet may be configured to permit removal of fluids (such as liquids, optionally by-products) flowing through said primary tube. In the event more than one outlet is provided, these may be distributed along the primary tube, for example evenly distributed along the primary tube with a regular interval between each said outlet.

The apparatus may be suitable for implementing the flow process for oxidising a substrate set out above. The process may involve mixing a substrate with catalase and one or more further enzymes to form a mixture and providing a flow of said mixture through the primary tube. Hydrogen peroxide may be provided to the flow of mixture through secondary tubing to form a reaction mixture.

There is also provided a process for the formation of a mono- or diester of furandicarboxylic acid from furandicarboxylic acid, the process comprising mixing furandicarboxylic acid, an alcohol and a catalyst, wherein the furandicarboxylic acid is obtained by a process for oxidising a substrate as defined above.

The furandicarboxylic acid may be 2,5-furandicarboxylic acid (2,5-FDCA).

The alcohol may comprise methanol and/or ethanol.

Any suitable esterification catalyst may be used. Suitable catalysts include organic acids or inorganic acids, such as mineral acids. Typical organic acids include acetic acid, trifluoroacetic acid or formic acid; typical inorganic acids include hydrochloric acid and sulfuric acid.

There is also provided a process for the formation of a copolymer comprising the copolyester of:
(a) furandicarboxylic acid (FDCA) and/or a mono- or diester of furandicarboxylic acid; and
(b) at least one diol;
wherein the process comprises polymerising components (a) and (b), wherein the furandicarboxylic acid is obtained by a process for oxidising a substrate as defined above, and/or wherein the mono- or diester of furandicarboxylic acid is obtained by a process for the formation of a mono- or diester of furandicarboxylic acid as defined above.

Such copolymers may exhibit properties that are similar to PBAT, such as being flexible, tough and biodegradable, and can be used either as a replacement for PBAT or in combination with PBAT.

The furandicarboxylic acid may be 2,5-furandicarboxylic acid (2,5-FDCA).

The furandicarboxylic acid (FDCA) or a mono- or diester of furandicarboxylic acid and diol can be referred to as monomers.

The copolymers of the invention may be a block copolymer, alternating copolymer, periodic copolymer, statistical copolymer or random copolymer. The copolymer may be a random copolymer.

Furandicarboxylic acids (FDCA) and mono- or diester of furandicarboxylic acids that are of particular interest are 2,5-FDCA, 2,5-FDCA-2-methyl ester, 2,5-FDCA dimethyl ester, 2,5-FDCA-2-ethyl ester, 2,5-FDCA diethyl ester and combinations thereof. The diester may be 2,5-FDCA diethyl ester.

The furandicarboxylic acid (FDCA) may be provided according to the processes for the oxidation of the substrate as defined above. The mono- or diester of 2,5-furandicarboxylic acids are provided according to the process for the formation of a mono- or diester of 2,5-furandicarboxylic acid as defined above.

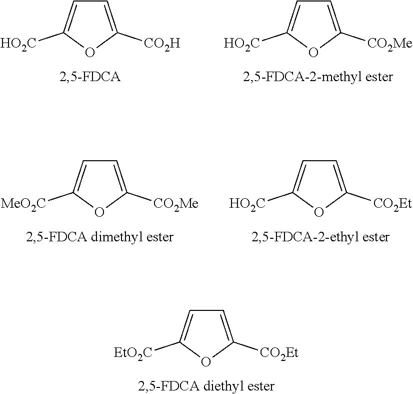

The at least one diol may comprise:

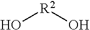

wherein R₂ is a straight, branched or cyclic C₂ to C₁₀ alkylene.

In a particular feature, the alkylene group present in the diol is unbranched.

In a preferred feature, the diol is 1,2-ethanediol, 1,4-butanediol, or combinations thereof, optionally 1,4-butanediol. Copolymers formed from 1,4-butanediol or 1,2-ethanediol may exhibit properties that are similar to PBAT, as discussed above.

The process may be for the formation of a copolymer that is the polymerisation product of
(A) 2,5-furandicarboxylic acid (2,5-FDCA) or a mono- or diester of 2,5-furandicarboxylic acid; and
(B) 1,2-ethanediol, 1,4-butanediol, or a combination thereof.

The copolymer may comprise the copolyester of:
(a) at least one furandicarboxylic acid (FDCA) or a mono- or diester of furandicarboxylic acid;
(b) at least one diol; and
(c) at least one dicarboxylic acid or a mono- or diester derivative thereof.

The dicarboxylic acid, or mono- or diester derivative thereof, may comprise an aliphatic dicarboxylic acid, or mono- or diester derivative thereof; heteroaromatic aromatic dicarboxylic acid, or mono- or diester derivative thereof; and/or an aromatic dicarboxylic acid, or mono- or diester derivative thereof.

The aliphatic dicarboxylic acid or a mono- or diester derivative thereof may comprise an unbranched alkylene group.

Aliphatic dicarboxylic acids or mono- or diester derivatives thereof that are of particular interest are adipic acid (hexanedioic acid), adipic acid monomethyl ester, adipic acid dimethyl ester, adipic acid monoethyl ester, adipic acid diethyl ester, succinic acid (butanedioic acid), succinic acid monomethyl ester, succinic acid dimethyl ester, succinic acid monoethyl ester, succinic acid diethyl ester, or combinations thereof, with adipic acid diethyl ester being particularly preferred. Copolymers formed from adipic acid or a mono or diester derivative thereof may exhibit properties that are similar to PBAT, as discussed above.

All combinations of furandicarboxylic acid, or a mono- or diester of furandicarboxylic acid; the at least one diol; and optionally the at least one dicarboxylic acid, or a mono- or diester derivative thereof, are contemplated in the present invention.

The skilled person would understand that additional monomers may be incorporated into the copolymers of the invention produced by the process for the formation of a copolymer as defined above. Therefore, the process may also be for the formation of a copolymer comprising the polymerisation product of components (a), (b) and optionally (c) and, in addition, a heteroaromatic diacid (or a mono- or diester derivative thereof), aromatic diacid (or a mono- or diester derivative thereof), heteroaromatic diol and/or aromatic diol.

Particular heteroaromatic and aromatic diacids that would be suitable for incorporation in the copolymers include pyridinedicarboxylic acids, such as 2,4-pyridinedicarboxylic acid (2,4-PDCA) and/or 2,5-pyridinedicarboxylic acid (2,5-PDCA), and terephthalic acid (or mono- or diester derivatives thereof).

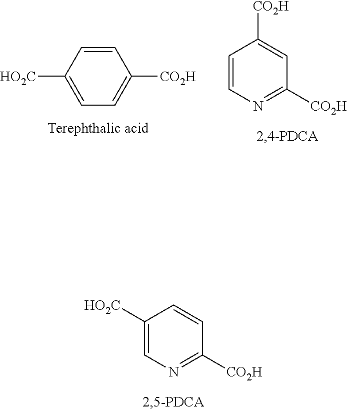 [SMILES: O=C(O)c1ccc(C(=O)O)cc1 ; O=C(O)c1ccnc(C(=O)O)c1 ; O=C(O)c1ccc(C(=O)O)nc1]

In a particular feature, there is provided a process for the formation of a copolymer comprising the polymerisation product of:
(a) 2,5-furandicarboxylic acid (2,5-FDCA), 2,5-FDCA-2-methyl ester, 2,5-FDCA dimethyl ester, 2,5-FDCA-2-ethyl ester, 2,5-FDCA diethyl ester or combinations thereof;
(b) 1,2-ethanediol, 1,4-butanediol, or combinations thereof; and,
(c) adipic acid, adipic acid monomethyl ester, adipic acid dimethyl ester, adipic acid monoethyl ester, adipic acid diethyl ester, succinic acid, succinic acid monomethyl ester, succinic acid dimethyl ester, succinic acid monoethyl ester, succinic acid diethyl ester, or combinations thereof
wherein the 2,5-furandicarboxylic acid is obtained by a process for oxidising a substrate as defined above, and/or wherein the mono- or diester of 2,5-furandicarboxylic acid is obtained by a process as defined in the process for the formation of a mono- or diester of furandicarboxylic acid as defined above.

It may be advantageous to use diester derivatives of furandicarboxylic acid and/or the dicarboxylic acid to form the copolymers of the invention. It may be particularly advantageous to use the same diester derivative of the furandicarboxylic acid and the dicarboxylic acid, i.e. furandicarboxylic acid diethyl ester and adipic acid diethyl ester.

The process may be for the formation of a copolymer that is the polymerisation product of
(A) 2,5-furandicarboxylic acid (2,5-FDCA) dimethyl ester or diethyl ester;
(B) 1,4-butanediol; and
(C) adipic acid dimethyl ester or diethyl ester,
   and in particular of
(A) 2,5-furandicarboxylic acid diethyl ester;
(B) 1,4-butanediol; and
(C) adipic acid dimethyl ester or diethyl ester.

A copolymer of the invention may be formed from about 1 to about 99 mol %, such as from about 10 to about 70 mol %, optionally from about 25 mol % to about 35 mol %, of 2,5-furandicarboxylic acid or a mono- or diester of furandicarboxylic acid.

A copolymer of the invention may be formed from about 1 to about 99 mol %, such as from about 20 to about 70 mol %, optionally from about 45 mol % to about 55 mol %, of diol. A copolymer of the invention may be formed from about 1 to about 98 mol %, such as from about 10 to about 70 mol %, optionally from about 25 mol % to about 35 mol %, of dicarboxylic acid a mono- or diester derivative thereof.

The above mol % values are based upon the total amount of furandicarboxylic acid or a mono- or diester of furandicarboxylic acid, diol, and optional dicarboxylic acid or mono- or diester derivative thereof.

When the copolymer consists essentially of furandicarboxylic acid or a mono- or diester of 2,5-furandicarboxylic acid, diol, and optionally dicarboxylic acids or mono- or diester derivatives thereof, the amount of diol may be about 50 mol % and the combined amount of 2,5-furandicarboxylic acid or a mono- or diester of furandicarboxylic acid and aliphatic dicarboxylic acids or mono- or diester derivatives thereof may also be about 50 mol %.

It will be understood by those skilled in the art that an excess of one of the monomers will typically result in polymer chains that terminate with that particular monomer.

The copolymers may be prepared by reacting together at least one furandicarboxylic acid or a mono- or diester of furandicarboxylic acid with at least one diol and at least one dicarboxylic acid or a mono- or diester derivative thereof simultaneously or concomitantly under standard conditions to form a copolymer. Such conditions include conditions suitable to perform, for instance, condensation reactions or transesterification reactions.

The reaction type is of course dependent upon the terminal groups of the monomer starting materials. Optionally, the polymers are prepared by melt polymerisation or solvent-based condensation reactions.

Those skilled in the art will understand the methods and conditions that may be used to react together the monomers to form a copolymer as a block copolymer, alternating copolymer, periodic copolymer, statistical copolymer or random copolymer. In an option, the process is for the formation of a random copolymer.

It is understood that the molar ratios of monomers used in the process may reflect the molar ratios of monomers as present in the resulting copolymer. This notwithstanding, it has been found to be advantageous to use an excess of at least one diol, in particular when a mono- or diester derivative of at least one furandicarboxylic acid and/or a mono- or diester of furandicarboxylic acid or at least one aliphatic dicarboxylic acid is used. Without wishing to be bound by theory, this may advantageously help to ensure that the terminal groups of the copolymers comprise an alcohol.

A suitable excess of at least one diol may be greater than about 5 mol %, such as greater than about 10 mol %, for instance greater than about 20 mol %, and optionally about 25 mol %, based upon the total amount of 2,5-furandicarboxylic acid or a mono- or diester of 2,5-furandicarboxylic acid and aliphatic dicarboxylic acid or mono- or diester derivative thereof in the reaction. Additional diol may be added during the process of the invention.

The formation of a copolymer may be carried out in the presence of a catalyst. Typical catalysts may contain a metal, such as a transition metal, or an organometallic catalyst, and a Lewis acid, with aluminium, tin, antimony, titanium, and their alkoxides being particularly preferred. Titanium(IV) tert-butoxide and titanium(IV) isopropoxide are exemplary catalysts.

The process for the formation of a copolymer may be carried out in the presence of a suitable solvent, for example water or an organic solvent such as ethyl acetate, toluene, tetrahydrofuran, diethyl ether, dioxane, dimethylformamide, dimethylsulfoxide, an alcohol (such as methanol or ethanol), or mixtures thereof (including biphasic solvent systems, such as a mixture of water and an organic solvent).

The process of the invention may be carried out “neat”, that is, no solvent is added to the reaction. The skilled person will understand that reacting together certain monomers (such as reacting together monomers comprising an ester group, i.e. an ethyl ester, with monomers comprising an alcohol group, in a transesterification reaction or condensation reaction) may form “solvent” (i.e. water or an alcohol, such as methanol or ethanol) as a result of the reaction. It is to be understood that the formation of a solvent during the reaction is not to be considered as solvent being added to the reaction. Such reactions are also considered to be carried out “neat”. It may however be advantageous to use ethyl acetate as a solvent when furandicarboxylic acid and/or a dicarboxylic acid is used in the process.

The process for the formation of a copolymer may be performed at any suitable reaction temperature, for instance at room temperature or an increased temperature. A feature of the invention is that the reaction may be carried out at one or more increased temperatures. That is, the reaction is heated to a first reaction temperature at which the reaction remains for a first length of time. After this time, the reaction temperature is changed (i.e. raised or lowered) to a second reaction temperature at which the reaction remains for a second length of time. The process of changing the reaction temperature may be subsequently repeated. Suitable temperatures include temperatures from about 60° C. to about 250° C., such as from about 90° C. to about 220° C., i.e. from about 110° C. to about 180° C.

Suitable times at which the reaction is held at a temperature are from about 1 hour to about 24 hours, such as from about 2 hours to about 19 hours, i.e. from about 3 hours or about 4 hours to about 17 hours.

The process for the formation of a copolymer may be performed at any suitable reaction pressure, for instance at atmospheric (or ambient) pressure or at an increased or reduced pressure.

The change in reaction pressure may coincide with a change in the reaction temperature. Those skilled in the art will understand that a change in pressure and/or temperature does not take immediately effect within a reaction. Therefore, when the change in reaction pressure coincides with a change in the reaction temperature, the changes are made at about the same time or over the same or similar time period.

The reaction pressure may be reduced over the course of the process of the invention. In particular, the process may be maintained at atmospheric pressure for a first time period, and then lowered to a reduced pressure for a second time period. The process of changing the reaction pressure may be subsequently repeated. Suitable reduced pressures include pressures from about 1 mbar to about 500 mbar such as from about 10 mbar to about 300 mbar i.e. from about 25 mbar to about 200 mbar.

In a particular feature of the process for the formation of a copolymers of the invention, the process is performed at 110° C. for 4 hours at atmospheric pressure, then at 180° C. for 17 hours at 200 mbar, and then at 180° C. for 3 hours at 25 mbar.

The polymerisation reaction may be mixed, i.e. stirred, to ensure that a homogeneous reaction mixture is formed. Mixing the reaction may ensure, for instance, that a homogeneous, random polymer is formed. As is known, the formation of a polymer may result in an increase in the viscosity of a reaction mixture. Those skilled in the art will appreciate that a suitable mixing device should be employed.

A copolymer that is obtained by the process may be purified or separated from the reaction mixture by standard techniques, for instance by precipitation and filtration, evaporation, chromatography, and/or evaporation of solvents.

In general, the process may be operated as a batch process or operated as a continuous process or flow process, and may be conducted on any scale.

The processes disclosed herein may have the advantage that the copolymers of the invention, or precursors thereof, may be produced in a high yield, in a high purity, in less time, in a more convenient form (i.e. easier to handle), at a low cost, and/or from renewable sources. The processes may be considered “green” or “clean” and therefore have environmental benefits for both the processes and the copolymers of the invention.

There is also provided a copolymer obtainable by the process for the formation of a copolymer as defined above.

The copolymer may have the formula:

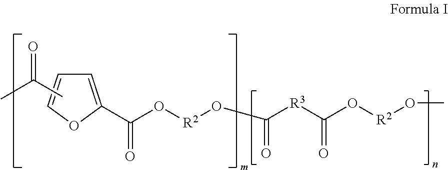

wherein R² and R³ are as defined above, and m and n are integers greater than zero.

The copolymer may also be as illustrated in Formula IA

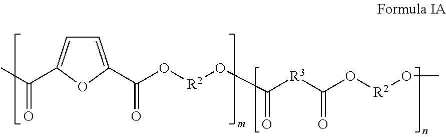

wherein R², R³, m and n are as defined above.

Particularly when carried out on an industrial scale, m may be from about 1 to about 600, such as from about 3 to about 550, for instance from about 5 to about 500, for example from about 7 to about 450, such as from about 10 to about 400, for example about 10 to about 350, such as from 10 to about 300, and n may be from about 1 to about 600, such as from about 3 to about 550, for instance from about 5 to about 500, for example from about 7 to about 450, such as from about 10 to about 400, for example about 10 to about 350, such as from 10 to about 300. In some circumstances, m and n may, independently, be at least about 50, such as at least about 75, optionally at least about 100, optionally at least about 120.

R² may be a C₂ to C₄ alkylene, and R³ may be a C₄ to C₆ alkylene.

It will be apparent to those skilled in the art that the nomenclature used in, for instance, Formula I does not denote the type of copolymer, i.e. a block copolymer, alternating copolymer, periodic copolymer, statistical copolymer or random copolymer. The copolymer of Formula I may be any copolymer type. However, the polymer may be a random copolymer.

The copolymer may also be as illustrated in Formula II

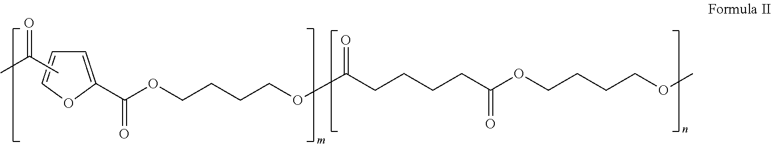

wherein m and n are as defined above.

The copolymer may also be as illustrated in Formula IIA

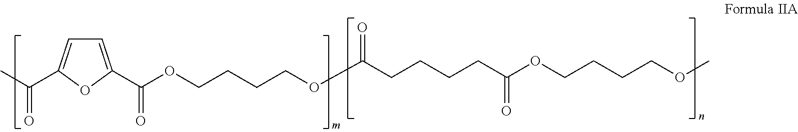

wherein m and n are as defined above.

A copolymer of the invention may have a molecular weight of from about 1,000 to about 500,000 gmol⁻¹, for example from about 10,000 to about 400,000 gmol⁻¹, such as from about 75,000 to about 300,000 gmol⁻¹, optionally from about 100,000 to about 150,000 gmol⁻¹, optionally from about 110,000 to about 130,000 gmol⁻¹, and optionally about 120,000 gmol⁻¹. Such copolymers have particularly useful properties.

The molecular weight of the copolymers was measured by Gel Permeation Chromatography (GPC) against a polystyrene standard set as per Example 10.

A copolymer of the invention may have at least one decomposition temperature within a range of from about 300° C. to about 450° C., and optionally from about 350° C. to about 400° C. Without wishing to be bound by theory, the decomposition temperature may relate to the decomposition of the copolymer backbone. Simultaneous Thermal Analysis (STA) was used to determine the decomposition temperature of copolymer samples under an inert (N₂) atmosphere as defined in Example 9.

A copolymer of the invention may have a first glass transition temperature (T_{g1}) within a range of from about −50° C. to about 0° C., and optionally from about −40° C. to about −20° C. The copolymer may have a second glass transition temperature (T_{g2}) within a range of from about 20° C. to about 60° C., and optionally from about 30° C. to about 50° C.

A copolymer of the invention may have a melting point (Tₘ) within a range of from about 60° C. to about 120° C., and optionally from about 80° C. to about 100° C.

Differential Scanning calorimetry (DSC) may suitably be used to determine the glass transition temperature (Tg) and the melting point (Tₘ), such as in accordance with Example 9. The glass transition temperature of the material may alternatively be measured using ASTM D3418-15 and/or ISO 11357-2:2013.

A copolymer of the invention may have a tensile strength in the range from about 1 MPa to about 50 MPa, such as from about 2 MPa to about 30 MPa, i.e. from about 3 MPa to about 15 MPa.

A copolymer of the invention may be stretched or elongated. The percentage elongation of the copolymer at its breaking point can range from about 1% to about 500%, such as from about 2% to about 300%, for example from about 3% to about 100% as based upon the original length of the copolymer.

A copolymer of the invention may have a Young's modulus in the range from about 10 MPa to about 500 MPa, such as from about 30 MPa to about 300 MPa, for example from about 50 MPa to about 150 MPa, i.e. from about 80 MPa to about 110 MPa.

Tensile strength, elongation and Young's modulus of the copolymers of the invention were measured as defined in Example 11.

Methods for testing the properties of copolymers, such as decomposition temperature, glass transition temperature, melting point, tensile strength etc. will be known to those skilled in the art.

A copolymer of the invention may be biodegradable and/or compostable. They may take less time to break down and be easier to recycle than current commercial polymers, such as PET and PBAT. Degradation may take place via a number of pathways including by hydrolysis and/or oxidation. Microorganisms, such as bacteria, yeasts, fungi, and also enzymatic processes also lead to biodegradation. For instance, enzymatic degradation of aliphatic polyesters including polyesters based upon succinic acid and aliphatic diols are known (see Tokiwa; Suzuki Nature 1977, 270, 76 to 78).

Products that conform to the EN13432:2000 or ASTMD6400-12 standards are deemed to be biodegradable and/or compostable, and may be considered to be compostable under “commercial” conditions with elevated temperatures (i.e. temperatures elevated above about 25° C.). Advantages of biodegradable and/or compostable products are that they can have a reduced carbon footprint, be more “environmentally friendly” (e.g. via reduction of waste to landfill), and/or be less reliant on fossil fuels for their production. The products of the invention may conform to the EN13432:2000 and/or ASTMD6400-12 standard.

It has been found that furandicarboxylic acid (such as 2,5-furandicarboxylic acid) represents a viable aromatic alternative to the use of terephthalic acid in polymers, such as PET and PBAT. Thus, copolymers comprising 2,5-furandicarboxylic acid may be useful as replacements for PET or PBAT, and minimise the environmental and economic impact of current commercial polymers.

There is also provided a polymer blend comprising a copolymer of the invention. A polymer blend may be defined as a macroscopically homogeneous mixture of two or more different species of polymer. For instance, the polymer blends may be binary, ternary, quaternary or higher polymer blends.

The copolymers of the invention may be blended with, for instance, polylactic acid (PLA), starch, cellulose acetate, polyhydroxybutyrate (PHB), isotactic polypropylene (PP), poly(butylene succinate), polybutylene succinate-co-adipate, polybutylene adipate-co-terephthalate, polyhydroxyalkanoate (e.g. polyhydroxy butyrate co-hexanoate or polyhydroxy butyrate co-valerate, such as poly(3-hydroxybutyrate-co-3-hydroxyvalerate)), poly-ε-caprolactone, poly(ethylene glycol), poly(ethylene oxide), and polymethyl methacrylate (PMMA). In an optional implementation, the copolymers of the invention are blended with PLA, starch and/or cellulose acetate.

The copolymers of the invention may be blended with fillers, (e.g. calcium carbonate, silica, talc, wollastonite, etc.) and/or compatibilisation agents (e.g. stearates, such as sorbitan monostearate (SMS), glycerol monostearate; fatty acid esters and/or amides).

A copolymer of the invention or polymer blends comprising the copolymer may take any physical form, for instance pellets, powders, sheets, fibres, or granules. It may be particularly advantageous for the copolymers or polymer blends to be pellets or granules to help processability or handling.

There is also provided an article comprising a copolymer of the invention or a polymer blend as described above. The term “article” is synonymous with an item or product. Such articles include articles currently made from plastics and in particular those made using materials comprising or consisting of PET and PBAT.

The copolymers of the invention may be used to form an article.

All features discussed above in respect of any of the processes, products or uses according to the invention discussed herein relate to all other processes, products or uses of the invention mutatis mutandis.

### DESCRIPTION OF THE FIGURES

FIG. 1 shows the ¹H NMR spectra for 2,5-polybutyrate adipate furandicarboxylate (2,5-PBAF), i.e. a copolymer of the invention.

FIG. 2 shows the 1H NMR spectra for polybutyrate adipate terephthalate (PBAT) (Comparative Example 8).

FIG. 3 shows the ¹H NMR spectra for commercial PBAT.

FIG. 4 shows the Simultaneous Thermal Analysis (STA) trace for 2,5-PBAF.

FIG. 5 shows the STA trace for PBAT (Comparative Example 8).

FIG. 6 shows the STA trace for commercial PBAT.

FIG. 7 shows Differential Scanning calorimetry (DSC) traces for 2,5-PBAF, PBAT (Comparative Example 8) and commercial PBAT.

FIG. 8 shows the Gel Permeation Chromatography (GPC) spectra for 2,5-PBAF.

FIG. 9 shows the Gel Permeation Chromatography (GPC) spectra for Comparative Example 8.

FIG. 10 shows the GPC spectra for commercial PBAT.

FIG. 11 shows the relative amount of DFF, FFCA and 2,5-FDCA as a function of time during the process described in Example 1.

FIG. 12 shows the relative amount of DFF, FFCA and 2,5-FDCA as a function of time during the process described in Example 2.

FIG. 13 shows the relative amount of DFF, FFCA and 2,5-FDCA as a function of time during the process described in Example 3.

FIG. 14 shows the relative amount of DFF, FFCA and 2,5-FDCA as a function of time during the process described Example 4.

FIG. 15 shows a comparison of the amount of 2,5-FDCA produced as a function of time during the processes described in Examples 1 to 4.

FIG. 16 shows that under the biodegradation test conditions outlined in Example 12, 2,5-PBAF result in a carbon loss of 29.3% after 40 days. The 90% level set for biodegradation in the test accounts for a +1-10% statistical variability of the experimental measurement, which one would expect virtually complete biodegradation in the composting environment of the test.

FIG. 17 shows that under the biodegradation test conditions outlined in Example 12, 2,5-PBAF loses carbon at a steady rate for over 60 days. The 90% level is as defined for FIG. 16 above.

FIG. 18 shows the attenuated total reflectance Fourier transform infrared spectra (ATR-FTIR) of 2,5-polybutyrate adipate furandicarboxylate (2,5-PBAF) using a Thermo Nicolet Nexus FT-IR spectrometer coupled with a Continuum IR microscope.

FIG. 19 shows the attenuated total reflectance Fourier transform infrared spectra (ATR-FTIR) of commercial PBAT using a Thermo Nicolet Nexus FT-IR spectrometer coupled with a Continuum IR microscope.

FIG. 20 is a perspective view of an apparatus for a continuous flow process of oxidising a substrate, as explained in Example 13.

FIG. 21 is a front view of an apparatus for a continuous flow process of oxidising a substrate, as explained in Example 14.

FIG. 22 shows calibration curves for quantitative gas chromatography (GC) and high-performance liquid chromatography (HPLC) analysis of hydroxymethylfurfural (HMF) and diformylfuran (DFF), as explained in Example 15.

FIG. 23 shows three GC traces for continuous-flow oxidation of 5-hydroxymethylfurfural, as explained in Example 15.

FIG. 24 shows results of a continuous run of HMF conversion using a trickle bed reactor, as explained in Example 16.

FIG. 25 shows a graphical representation of suitable flow velocity for use in the apparatus depicted in FIG. 20.

The following examples are merely illustrative examples of the invention described herein and are not intended to be limiting upon the scope of the invention.

### EXAMPLES

### General Experimental Information and Materials

The E. coli TP1000 mutant strain used for PaoABC expression is a derivative of MC4100 with a kanamycin cassette inserted in the mobAB gene region. E. coli xanthine dehydrogenase and catalase were sourced from Sigma-Aldrich. Starting materials were purchased from Alfa Aesar and Sigma-Aldrich and used as received. HPLC analysis was performed on an Agilent 1200 system equipped with a G1379A degasser, G1312A binary pump, a G1329 autosampler unit, a G1315B diode array detector and a G1316A temperature controlled column compartment. The columns used were Thermofisher Hypurity C18 (5 μm particle size, 4.6 mm diameter×250 mm), Thermofisher ODS Hypersil C18 (5 μm particle size, 4.6 mm diameter×250 mm) and Bio-Rad Aminex HPX-87H, 300 mm×7.8 mm pre-packed column. GC analysis was performed on an Agilent 7890A chromatograph using an Alltech SE-30, 30.0 m×320 μm×0.25 μm column. Conditions are indicated separately for each compound. ¹H NMR spectra were recorded on a Bruker Avance 400 or 500 without additional internal standard.

### Preparation of Enzymes

Galactose Oxidase Variant M₃₋₅ (GOase M₃₋₅)

GOase mutant M₃₋₅ (Escalettes; Turner J. ChemBioChem 2008, 9, 857-860) was transformed into E. coli BL21 Star™ (DE3) cells (Invitrogen) according to manufacturer's specifications. A single colony was picked from an overnight LB plate containing 1 μL of kanamycin of a 30 mg/mL stock solution per mL of agar and used to inoculate 5 mL LB medium supplemented with 5 μL kanamycin and grown overnight at 37° C. and 250 rpm. 500 μL of the overnight culture was used to inoculate 250 mL of an autoinduction medium (8ZY-4LAC) as described by Deacon and McPherson (Deacon; McPherson J. ChemBioChem. 2011, 12, 593-601) and supplemented with 250 μL of kanamycin in a 2-L-baffled Erlenmeyer flask. The cells were grown at 26° C. and 250 rpm for 60 hour. Cells were harvested by centrifugation at 6,000 rpm and 40° C. for 20 min and subsequently prepared for protein purification.

### Purification of GOase M₃₋₅

The cell pellet from a 250-mL-culture was resuspended in 30 mL lysis buffer containing 50 mM piperazine-N,N′-bis(2-ethanesulfonic acid) (PIPES), 25% sucrose (w/v), 1 mg mL⁻¹ lysozyme, 5 mM MnCl₂ and 1% Triton X-100 (v/v). The suspension was gently shaken at 4° C. for 20 min. Afterwards, cells were mechanically disrupted via ultrasonication (30 sec. on, 30 sec. off; 20 cycles) followed by ultracentrifugation (20,000×g, 30 min, 4° C.). The cleared crude extract was transferred into a flexible tubing (30 kDa cut-off), dialysed into buffer C (50 mM NaPi buffer, 300 mM NaCl, pH 8.0) for 12 hours at 4° C. and subsequently passed through a syringe filter with a 0.22 μm pore size. Protein purification was accomplished with a peristaltic tubing pump (Thermo Scientific) equipped with a 5-mL-Strep-Tag®-II column (GE Healthcare) pre-equilibrated with buffer C. After loading with crude extract, the column was washed with 5 column volumes of buffer C followed by protein elution with 70 mL of buffer D (50 mM NaPi buffer, 300 mM NaCl, 5 mM desthiobiotin, pH 8.0).

For copper-loading, GOase Mm-containing fractions were pooled and subsequently transferred into flexible dialysis tubing (30 kDa cut-off) and dialysed twice for 12 hours into buffer E (50 mM NaPi buffer saturated with CuSO₄, pH 7.4) at 4° C. Removal of excess CuSO₄ was attained by two cycles of dialysis into buffer E (without CuSO₄) for 12 hours at 4° C. and protein samples concentrated to approximately 3 mg/mL using a Sartorius Vivaspin 6 spin column (30 kDa mass cut-off). The protein samples were aliquoted and the aliquots were frozen in liquid nitrogen prior to storage at −80° C.

E. coli Perisplasmic Aldehyde Oxidase (PaoABC)

For PaoABC expression (Neumann et al. FEBS Journal 2009, 276, 2762-2774), the plasmid pMN100 derived from pTrcHisA (Invitrogen), containing the PaoABC genes with a His6 tag fused to the N-terminus of PaoA, was used. For heterologous expression in E. coli, pMN100 was transformed into E. coli TP1000 cells, containing a deletion in the mobAB genes responsible for Moco dinucleotide formation. One litre of LB supplemented with 1 mM sodium molybdate and 10 μM isopropyl thio-β-D-galactoside was inoculated with 2 mL of an overnight culture and incubated for 24 hours at 22° C. and 100 rpm. The cells were harvested by centrifugation at 4,000×g for 15 min.

### Purification of PaoABC

The cell pellet was resuspended in 8 volumes of 50 mM sodium phosphate, 300 mM NaCl, pH 8.0, 10 mM imidazole and cell lysis was achieved by sonication (MSE Soniprep) with cooling on ice (20 bursts of 20 s on/off at 14 u). After addition of DNase I, the lysate was incubated for 30 min. After centrifugation at 17,000×g for 25 min the supernatant was filtered through 0.45 and 0.2 μM membranes before loading onto Ni₂-nitrilotriacetic agarose (HiTrap 1 mL column (GE Healthcare)). The column was washed with 2 column volumes of 10 mM imidazole, 50 mM sodium phosphate, 300 mM NaCl, pH 8.0, followed by a wash with 10 column volumes of the same buffer with 20 mM imidazole. His-tagged PaoABC was eluted with 20 mL of 100 mM imidazole in 50 mM sodium phosphate, 300 mM NaCl, pH 8.0. Fractions containing PaoABC were buffer exchanged into 50 mM Tris, 1 mM EDTA, pH 7.5. The yield of protein was about 13 mg/L of E. coli culture.

### Xanthine Dehydrogenase Variants E232V and E232VR310 (XDH E232V, XDH E232VR310)

For expression of XDH mutants, the plasmid pSL207 derived from pTrcHisA (Invitrogen), containing the xdh genes with a His6 tag fused to the N-terminus of XDHA, was used. For heterologous expression in E. coli, pSL207 was transformed into E. coli TP1000 cells, containing a deletion in the mobAB genes responsible for Moco dinucleotide formation. The enzyme was expressed in 500-mL-cultures of TP1000 cells carrying plasmid pSL207 grown at 30° C. in LB medium supplemented with 150 μg/mL ampicillin, 1 mM molybdate, and 0.02 mM isopropyl-D-thiogalactopyranoside until the OD₆₀₀ₙₘ=1. This culture was then transferred to a bottle containing 8 L of supplemented LB medium and subsequently grown at 30° C. for 18 to 20 hours. Cells were harvested by centrifugation at 5000×g at 4° C. and subsequently prepared for protein purification.

### Purification of XDH E232V and XDH E232VR310

The cell pellet was resuspended in eight volumes of 50 mM sodium phosphate, 300 mM NaCl, pH 8.0, and cell lysis was achieved by several passages through a French press. After addition of DNase I, the lysate was incubated for 30 min. After centrifugation at 17,000×g for 25 min, imidazole was added to the supernatant to a final concentration of 10 mM. The supernatant was mixed with 2 mL of Ni₂-nitrilotriacetic agarose (Qiagen) per litre of cell growth, and the slurry was equilibrated with gentle stirring at 4° C. for 30 min. The slurry was poured into a column, and the resin was washed with two column volumes of 10 mM imidazole, 50 mM sodium phosphate, 300 mM NaCl, pH 8.0, followed by a wash with ten column volumes of the same buffer with 20 mM imidazole. His-tagged XDH was eluted with 100 mM imidazole in 50 mM sodium phosphate, 300 mM NaCl, pH 8.0. Fractions containing XDH were combined and dialyzed against 50 mM Tris, 1 mM EDTA, 2.5 mM dithiothreitol, pH 7.5. The dialyzed sample was applied to a Q-Sepharose fast protein liquid chromatography column and eluted with a linear gradient of 0-250 mM NaCl. To the pool of fractions containing XDH, 15% ammonium sulphate was added, and the protein was then applied to a phenyl-Sepharose column equilibrated with 50 mM Tris, 1 mM EDTA, 2.5 mM dithiothreitol, 15% ammonium sulphate, pH 7.5. XDH E232V was eluted from the column with a linear gradient of from 15 to 0% ammonium sulphate. During purification, fractions were monitored using SDS-PAGE, whereas enzyme activity was measured spectrophotometrically as described above.

### Screening of Xanthine Oxidoreductases for Oxidation of HMF, DFF and FFCA

XORs were screened using potassium phosphate buffer (50 mM, pH 7.6), 3 μL 0.1 M HMF (in MeCN), 30 μL 0.01 M DCPIP (aq.) final volume 300 μL, 36° C. When DCPIP was the oxidant, activity was determined by the colour change from blue to colourless; when O₂ was the oxidant, activity was detected using NBT assay (Agarwal; Banerjee Open Biotech J. 2009, 3, 46-49). The results can be found in Table 1.

TABLE 1
Screening of xanthine oxidoreductases

| Enzyme | HMF | DFF | FFCA | Oxidant |
|---|---|---|---|---|
| E. coli XDH^{a} | Active | — | — | O₂ |
| XDH E232V^{b} | Active | Active | Active | DCPIP |
| XDH E232V R310^{c} | Active | Active | Active | DCPIP |
| PaoABC^{d} | Active | Active | Active | O₂ |

^{a}E. coli XDH (1.1 mg/mL);
^{b}XDH E232V (25.4 mg/mL);
^{c}XDH E232V/R310M (23 mg/mL);
^{d}PaoABC (13.3 mg/mL);
^{e}PaoABC (13.3 mg/mL).

### Example 1—No Initial Shaking of Buffer

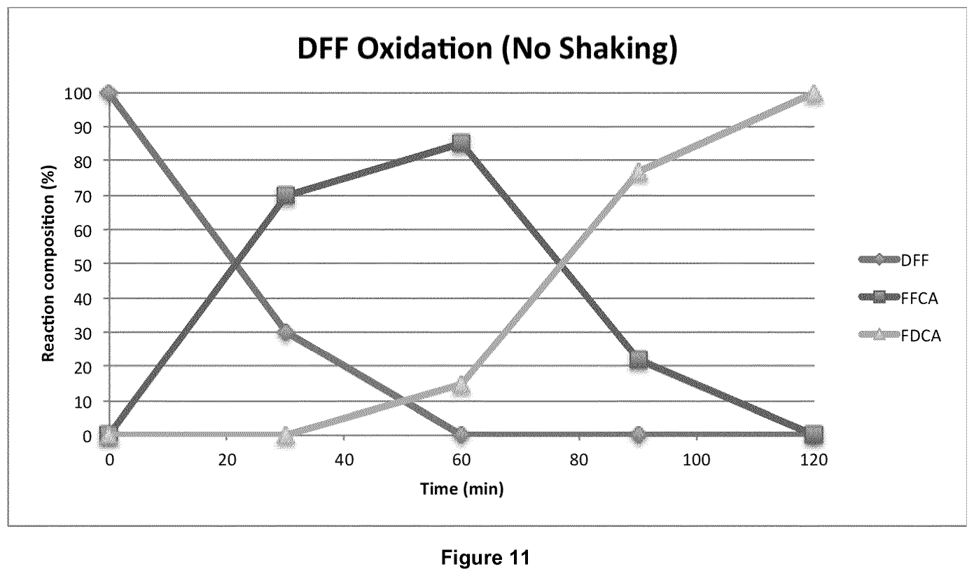

To 490 μL of 0.2 M KPi buffer pH 7.0 was added DFF (final concentration 100 mM) and 1 mg catalase. 10 μL of a 100 μM PaoABC was then added and the reaction was left in a shaking incubator. 5 μL of the reaction mixture was extracted, diluted with 80 μL water and quenched with 15 μL 1 M HCl. The aliquots were analysed by RP HPLC. The results are shown in FIG. 11.

### Example 2—Initial Shaking of Buffer

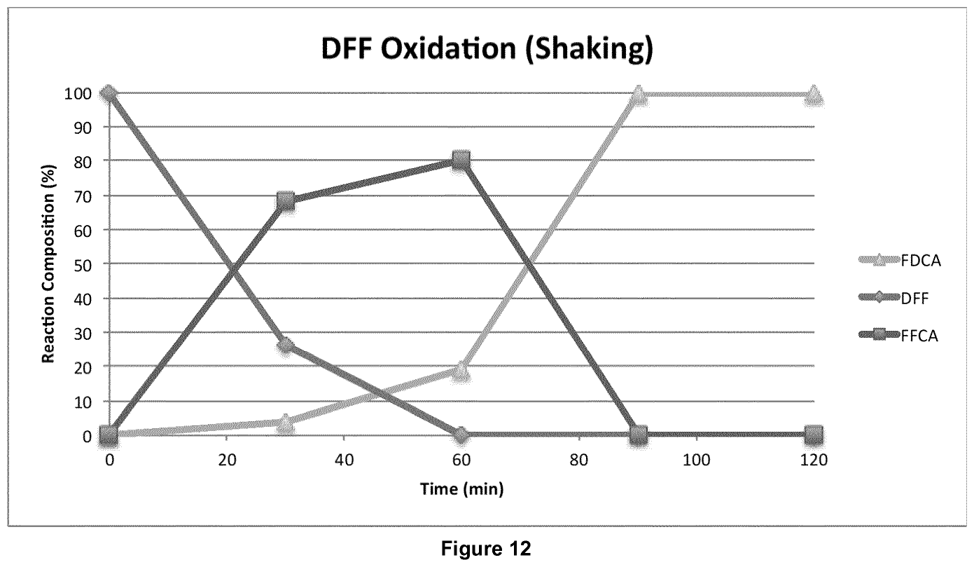

To 490 μL of 0.2M KPi buffer pH 7.0 was added DFF (final concentration 100 mM) and 1 mg catalase. The Eppendorf was vigorously shaken. 10 μL of a 100 μM PaoABC was then added and the reaction was left in a shaking incubator. 5 μL of the reaction mixture was extracted, diluted with 80 μL water and quenched with 15 μL 1M HCl. The aliquots were analysed by RP HPLC. The results are shown in FIG. 12.

### Example 3—Oxygen Sparged Buffer

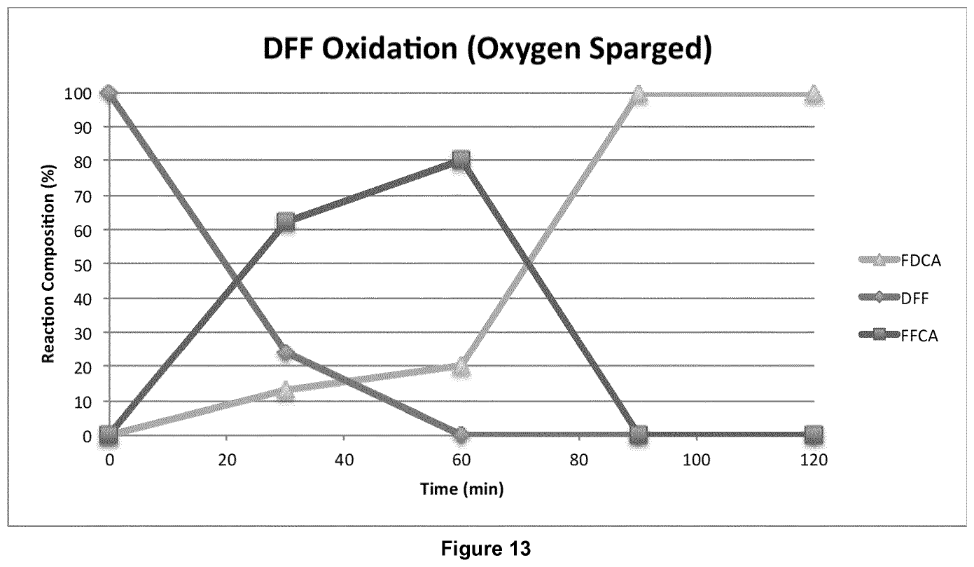

To 490 μL of 0.2 M KPi pH 7.0 (previously sparged with compressed air (HPLC filter) for 5 hours) was added DFF (final concentration 100 mM) and 1 mg catalase. 10 μL of a 100 μM PaoABC was then added and the reaction was left in a shaking incubator. 5 μL of the reaction mixture was extracted, diluted with 80 μL water and quenched with 15 μL 1 M HCl. The aliquots were analysed by RP HPLC. The results are shown in FIG. 13.

### Example 4—Periodic Hydrogen Peroxide Addition

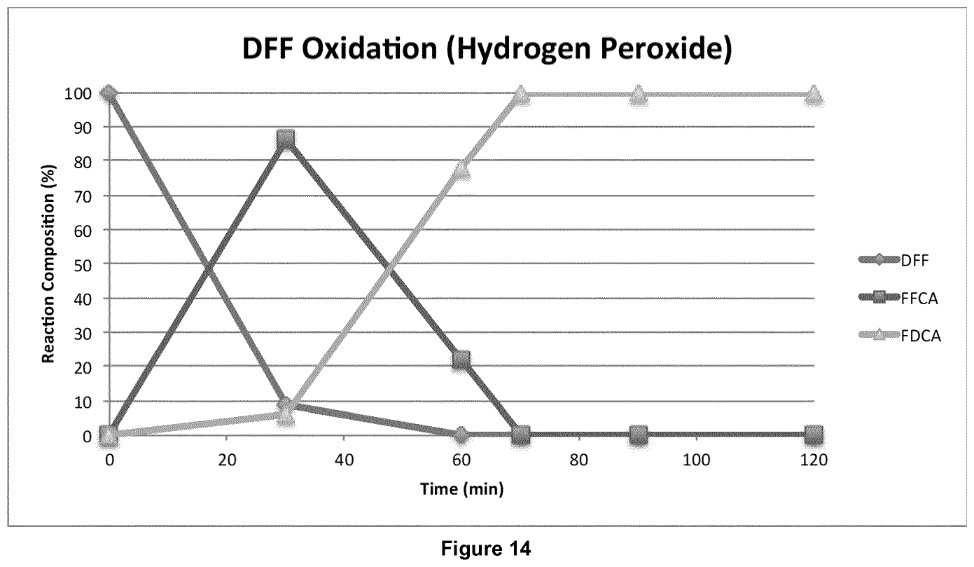

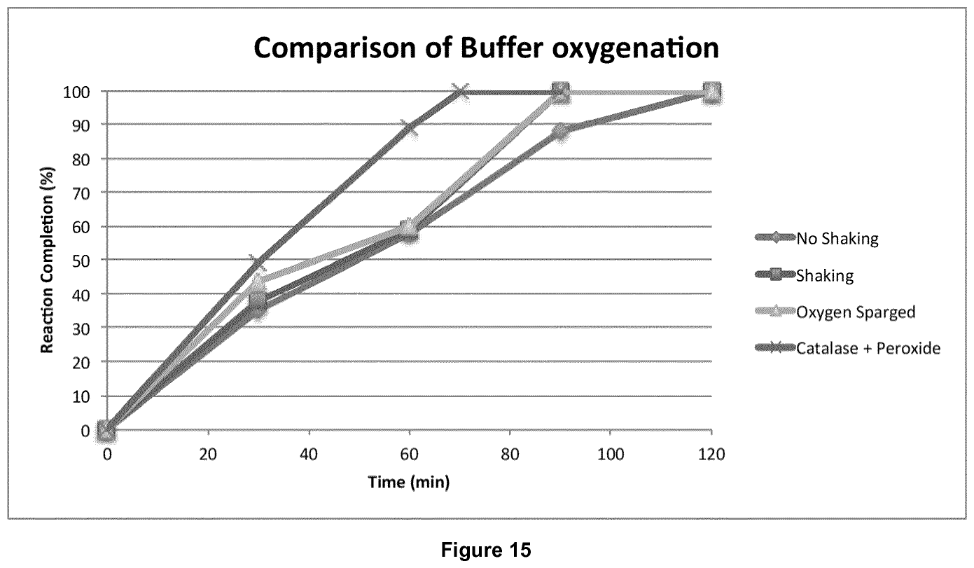

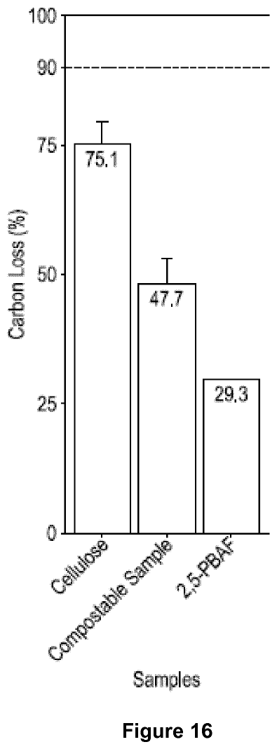

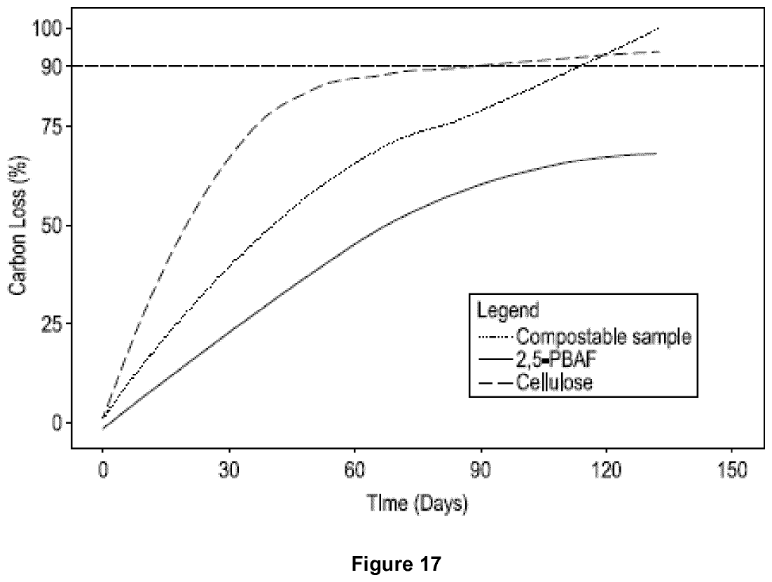

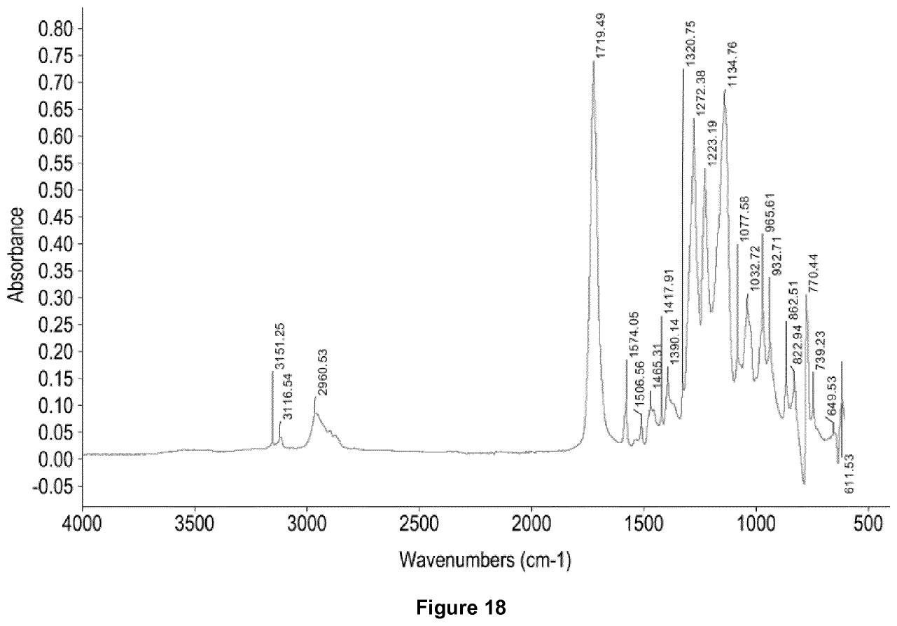

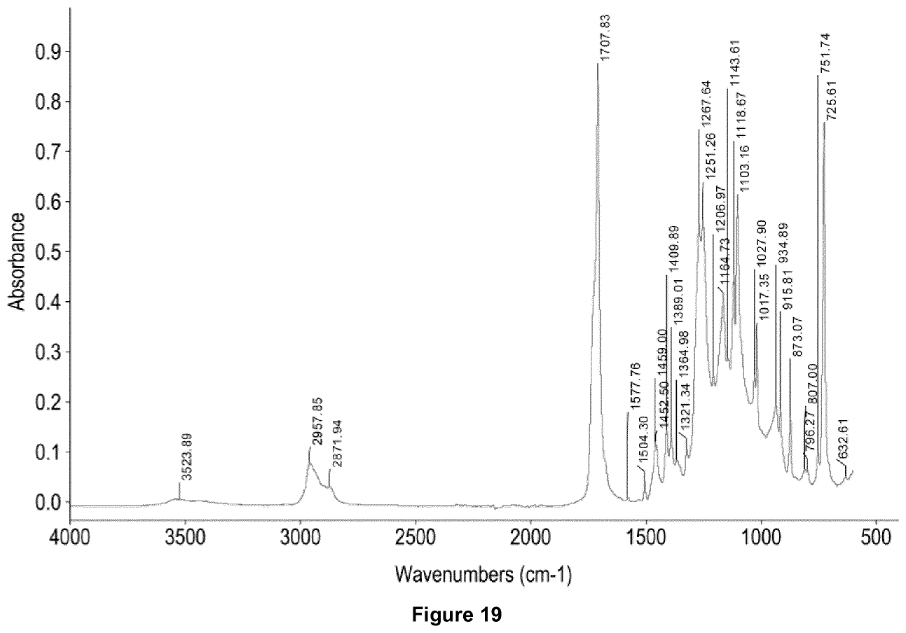

To 490 μL of 0.2 M KPi pH 7.0 was added 0.05 mmol DFF and 1 mg catalase. 10 μL of a 100 μM PaoABC was then added and the reaction was left in a shaking incubator. About 0.0003 mmol H₂O₂ was added every 15 minutes (1 μl of a 1% solution). 5 μL of the reaction mixture was extracted, diluted with 80 μL water and quenched with 15 μL 1 M HCl. The aliquots were analysed by RP HPLC. The results are shown in FIGS. 14 and 15.

### Example 5A—Entrapment of PaoABC in SiO₂ Hydrogel

Tetramethyl orthosilicate (TMOS) (0.450 g) was placed in a small vial, cooled in an ice bath, and stirred at about 600 rpm. HCl (108 μL, 2.44 mM) was added, and the solution was stirred for 10 min. The solution was adjusted to pH 5.1 by adding 60 μL of 20 mM sodium phosphate buffer (pH 7.4). In a separate small vial, 1 mg of PaoABC, 540 μL of 10 mM 4-(2-hydroxyethyl)-1-piperazineethanesulfonic acid (HEPES) (sodium salt) buffer solution (pH 7.5), and, when polymer was added, 60 μL of 20 mg/mL aqueous PVI or PEI were mixed. The PaoABC-containing solution was added to the TMOS-containing solution, and the resulting mixture was stirred for 1 min. A vacuum was applied to the stirred mixture until a gel formed. The vacuum was released, and the gel was rinsed with 2 mL of distilled water three times. The gel was then soaked in 2 mL of distilled water overnight at 4° C. The water was removed from the vial, and the gel was allowed to dry at room temperature overnight in a vial. The dried gel was first collected and then ground to a powder with a mortar and pestle. 750 mg of hydrogel was produced with 1 mg of PaoABC, as determined by Bradford assay of the supernatant.

### Example 5B—Entrapment of PaoABC in Ni-Sepharose

30 mL binding buffer (400 mM NaCl, 20 mM Imidazole, 50 mM KPi pH 7.0), 3 g of PaoABC CFE (cell free extract) and 5 g Ni⁺² 5 g of resin was stirred for 40 minutes. The slurry was centrifuged at 500 RPM and the supernatant removed. This was repeated twice with washing with binding buffer.

### Example 5C—Entrapment of PaoABC in Eupergit EC

35 μl of PaoABC was dissolved in 1 mL 1M KPi buffer with a pH as specified in Table 2. The concentration of PaoABC before immobilisation was recorded. 10 mg of Eupergit EC was added and the mixture was left in a shaking incubator at 150 rpm and 25° C. for 24 hours. The amount of enzyme absorbed onto the resin was determined by UV spectroscopy (428 nm). Blocking buffer (0.2M ethanolamine in 0.1M KPi pH 7) was added and the mixture was left to shake for 30 minutes. The immobilised enzyme was filtered through a sinter funnel and washed with KPi buffer pH 7 and then KPi buffer pH 7 with 1M NaCl. The immobilised beads were tested for activity.

TABLE 2

| | | | Enzyme | |
|---|---|---|---|---|
| | Immobilisation | Enzyme | Immobilized | Conversion^{[a]} |
| Entry | pH | (mg/mL) | (mg) | (%)^{[b]} |
| 1 | 6 | 0.85 | 0.7 | 44 |
| 2 | 7 | 1 | 0.97 | 48 |
| 3 | 8 | 0.94 | 0.8 | 60 |

^{[a]}Reaction conditions: 0.1 mg PaoABC (on resin), 2.5 μl benzaldehyde, 500 μl 100 mM KPi pH 7.6, 37° C., 5 Hr
^{[b]}Conversion calculated by RP HPLC

### Example 5D—Entrapment of PaoABC in Eupergit CM

70 μL (2 mg) of paoABC was dissolved in 1 mL 1M KPi buffer pH 7. The concentration before immobilisation was recorded. 200 mg of Eupergit EC was added and left in a shaking incubator on 150 rpm at 25° C. for 24 Hr. The amount of enzyme absorbed onto the resin was determined by UV spectroscopy (428 nm). After this time the pH was increased to pH 8.5 to facilitate multipoint attachment to the resin (5 hr). Blocking buffer (3M Glycine pH 8.5) was added and left shake for 5 hours. The immobilised enzyme was filtered through a filter paper and washed with 100 mM KPi Buffer pH 7 and, 100 mM KPi buffer pH 7 with 1M NaCl. The immobilised beads were tested for activity.

| | | | | Conversion |
|---|---|---|---|---|
| Entry | Resin | Amount | Run | (%) |
| 1 | Eupergit CM | 50 mg | 1 | 100 |
| 2 | ″ | ″ | 2 | 100 |

Reaction conditions: 100 mM KPi phosphate buffer pH 7.0, 100 mM DFF, 5 hr, 37° C.

### Example 6

### Synthesis of 2,5-diethyl-2,5-furandicarboxylate

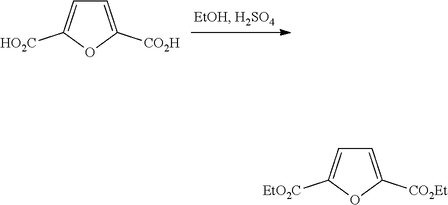 [SMILES: O=C(O)c1ccc(C(=O)O)o1 ; CCOC(=O)c1ccc(C(=O)OCC)o1]

2,5-furandicarboxylic acid (25.18 g; 160 mmol) was added to ethanol (1,800 mL). Aqueous sulfuric acid (1.32 mL) was added. The mixture was heated at reflux (about 78° C.) for 67 hours, during which time water was removed from the reaction by the use of a Dean-Stark apparatus. The reaction progress was monitored using NMR spectroscopy. After the 2,5-diethyl-2,5-furandicarboxylate had been formed in >97% purity by NMR, the reaction mixture was allowed to cool to ambient temperature and was extracted with 2-methyltetrahydrofuran. The combined organic layers were washed with a saturated aqueous brine solution and deionised water, and dried (MgSO₄). The organics were filtered and the volatiles were removed in vacuo to afford the title compound (26.77 g; 130 mmol; >98% conversion).

### Example 7

### General Methodology for the Formation of Copolymers

A 250 mL flange flask with 5 quick-fit ports was used in connection with a Dean-Stark apparatus. Stirring was achieved via a magnetic stirrer using a large precious metal stirrer bar. The rates of stirring were gradually decreased from the initial 400 rpm down to 200 rpm to avoid issues as a result of the increasing viscosity of the reaction mixture. All reagents were added to the reactor and warmed to 110 to 130° C. as described below to allow total melting and achieve miscibility. A flow of N₂ gas was applied for 20 minutes to purge the reagents and reactor of oxygen. The temperature was then increased to the desired point as stated below. After a further four hours of very low N₂ flow the gas line was removed, the Dean-Stark drained and a vacuum pump turned on, initially at a low vacuum (˜200 mbar) but slowly increased as stated below.

### Synthesis of 2,5-polybutyrate adipate furandicarboxylate (2,5-PBAF)

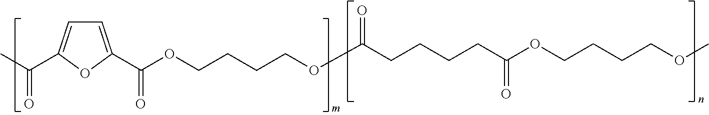

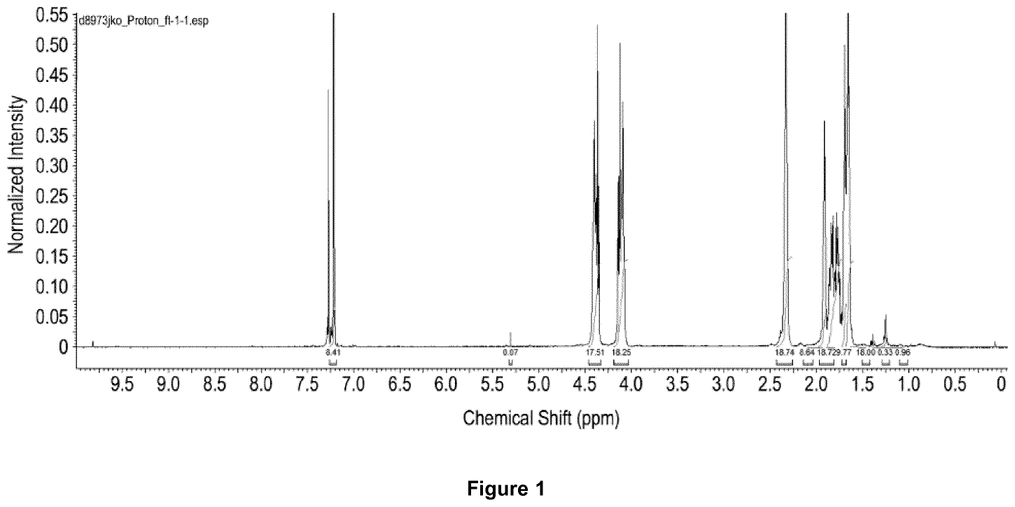

2,5-Diethyl-2,5-furandicarboxylate (21.20 g; 100 mmol), diethyl adipate (20.23 g; 100 mmol), 1,4-butane diol (22.53, 250 mmol) and T titanium(IV) tert-butoxide (0.77 mL; cat.) were combined. The reaction mixture was heated at 110° C. for 4 hours at atmospheric pressure with stirring at 400 rpm, 180° C. for 17 hours at 200 mbar and 350 rpm, and at 180° C. for 3 hours at 25 mbar and 250 rpm. The polymer was formed (37.20 g). The ¹H NMR spectra for 2,5-PBAF can be found at FIG. 1.

The molar ratio of 2,5-furandicarboxylate:adipate was determined by ¹H NMR to be 0.90:1. The molecular weight of the 2,5-PBAF was estimated using end-group analysis, wherein the ratio of end groups to those of the bulk polymer were calculated using ¹H NMR to give the number of constitutional repeating units (CRU), which was estimated to be 20.71. One ideal CRU is 410.43 gmol⁻¹. Therefore, the molecular weight of the 2,5-PBAF was estimated to be 8,497.5 gmol⁻¹.

### Comparative Example 8

### Synthesis of Polybutyrate Adipate Terephthalate (PBAT)

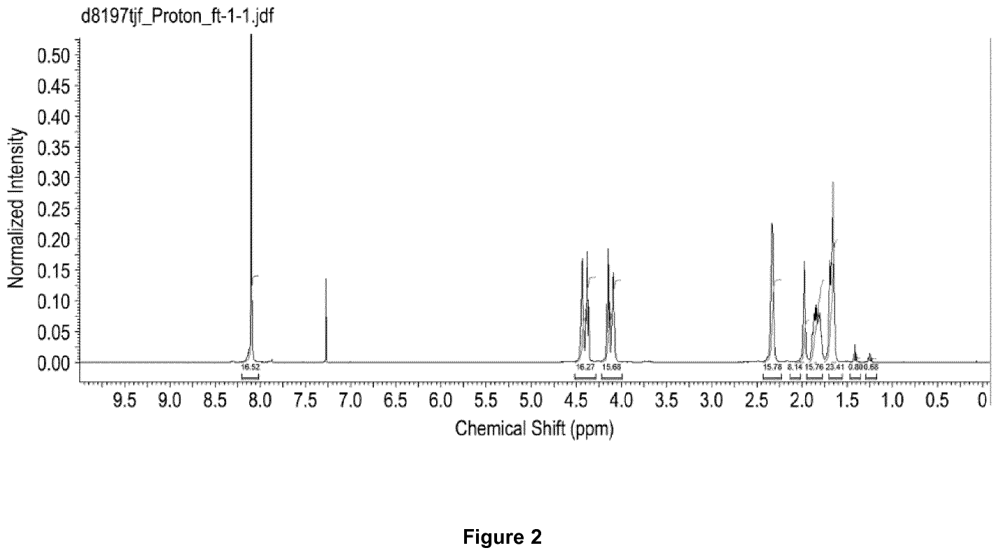

Diethyl terephthalate (22.22 g; 100 mmol), diethyl adipate (20.23 g; 100 mmol), 1,4-butane diol (22.73, 230 mmol) and titanium(IV) tert-butoxide (0.77 mL; cat.) were combined. The reaction mixture was heated at 130° C. for 2 hours at atmospheric pressure with stirring at 400 rpm, 180° C. for 2 hours at atmospheric pressure and 400 rpm, 180° C. for 17 hours at 200 mbar and 350 rpm, and at 180° C. for 3 hours at 25 mbar and 250 rpm. The copolymer was formed (40.51 g). The ¹H NMR spectra for PBAT can be found at FIG. 2.

The molecular weight of the PBAT was estimated by ¹H NMR using end-group analysis as described for 2,4-PBAP. The molar ratio of terephthalate:adipate was determined to be 1.047:1. The number of CRUs was estimated to be 16.4. One ideal CRU is 420.45 gmol⁻¹. Therefore, the molecular weight of the PBAT was estimated to be 6,893 gmol⁻¹.

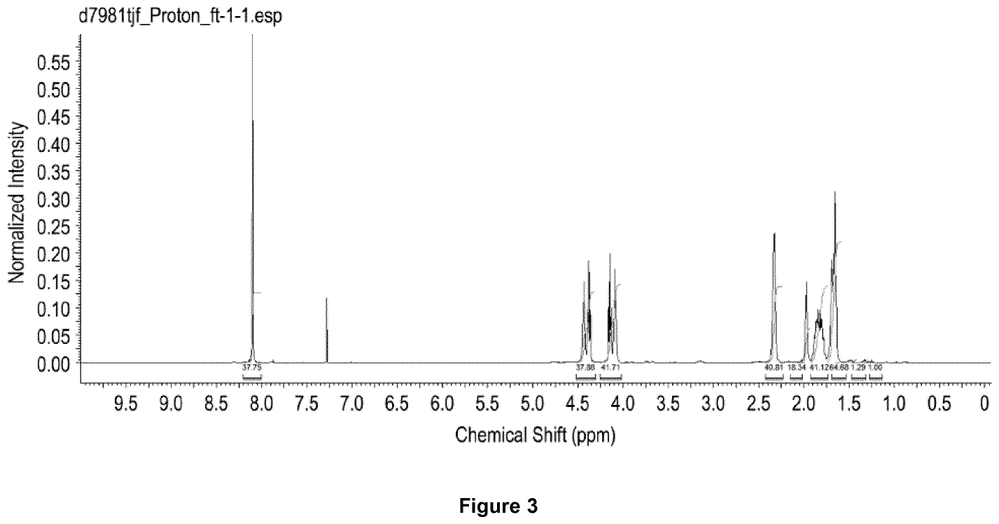

PBAT is available commercially under a range of trade names. The molecular weight of one particular commercial PBAT was estimated by ¹H NMR using end-group analysis as described for 2,4-PBAP. The molar ratio of terephthalate:adipate was determined to be 0.93:1. The number of CRUs was estimated to be 25.7. One ideal CRU is 420.45 gmol⁻¹. Therefore, the molecular weight of the commercial PBAT was estimated to be 10,809 gmol⁻¹. The ¹H NMR spectra for commercial PBAT can be found at FIG. 3.

### Example 9

### Thermal Analysis of Polymers Using (STA and DSC)

The thermal stability of cured copolymer was analysed using Simultaneous Thermal Analysis (STA) using a Stanton Redcroft STA 625. Approximately 10-20 mg of copolymer was heated from ambient temperature to 625° C. at a heating rate of 10° C. min⁻¹ under nitrogen. The decomposition may be that of the copolymer backbone. The results can be found in Table 3.

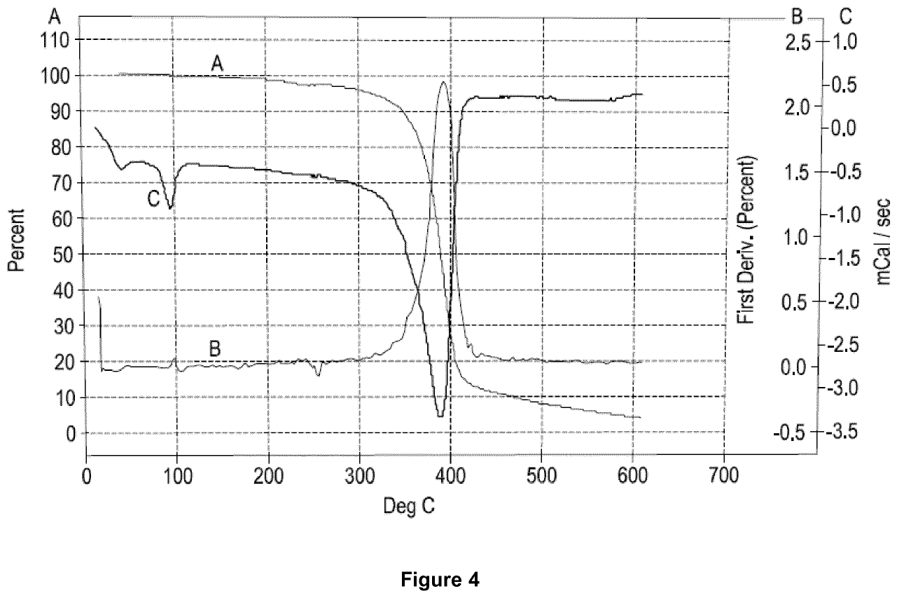

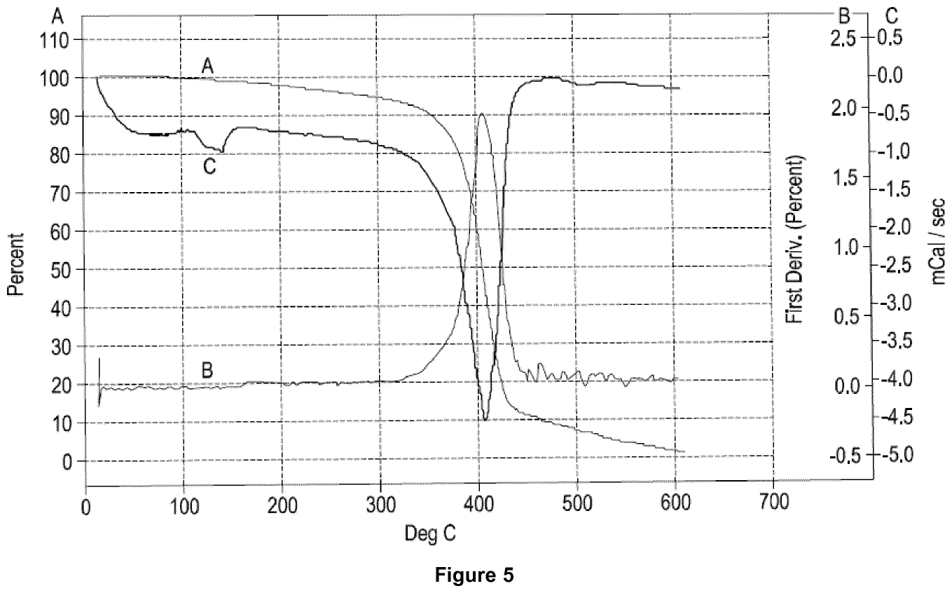

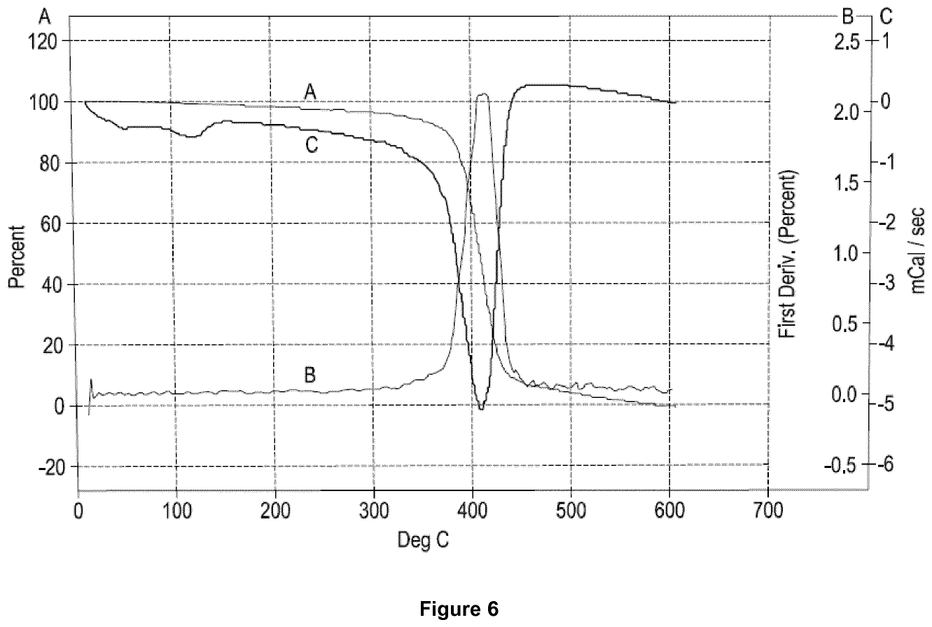

TABLE 3
STA analysis of polymers

| | Temperature of | Temperature of | |
|---|---|---|---|
| | 5 wt % loss | decomp. | |
| Copolymer | ° C. | ° C. | STA trace |
| 2,5-PBAF | 315.0 | 391.7 | FIG. 4 |
| Comparative Example 8 | 289.5 | 406.0 | FIG. 5 |
| Commercial PBAT | 341.5 | 409.5 | FIG. 6 |

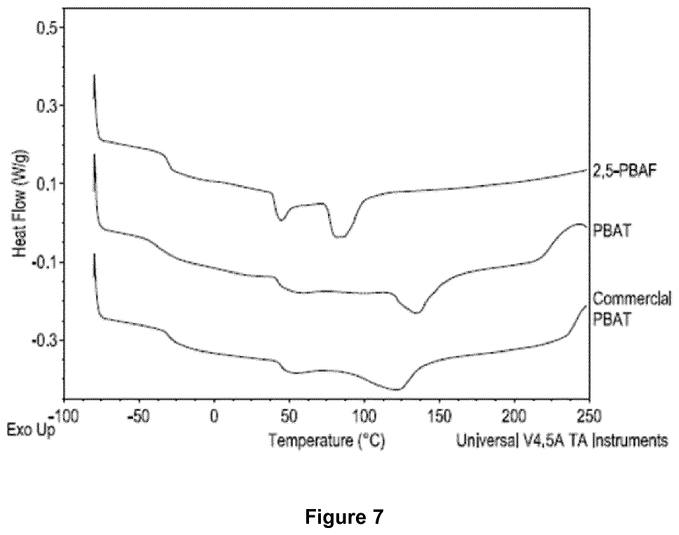

The glass transition temperature (T_{g}) and melting point (Tₘ) of the copolymers were obtained by Differential Scanning calorimetry (DSC) analysis using a TA Instruments Q2000 DSC. Indium was used as the standard to calibrate the temperature and heat capacity. Copolymer samples (7-10 mg) were sealed in Tzero aluminum hermetic DSC pans. The method was carried out under a constant flow of dry nitrogen of 50 mL/min, at 10° C./min over a temperature range of −80° C. to 250° C. The results can be found in Table 4. The DSC traces can be found at FIG. 7.

TABLE 4
DSC analysis of copolymers

| | T_{g1} | T_{g2} | Tₘ |
|---|---|---|---|
| Copolymer | ° C. | ° C. | ° C. |
| 2,5-PBAF | −30.4 | 40.5 | 87.2 |
| Comparative Example 8 | −39.5 | 42.4 | 134.6 |
| Commercial PBAT | −30.1 | 45.4 | 122.2 |

### Example 10

The molecular weight (Mₙ and M_{w}) and polydispersity (Pdᵢ) data as generated by GPC can be found in Table 5. GPC was conducted on an Agilent SECurity GPC System 1260 Infinity using THF as the solvent, a polystyrene standard, and a light scattering detector.

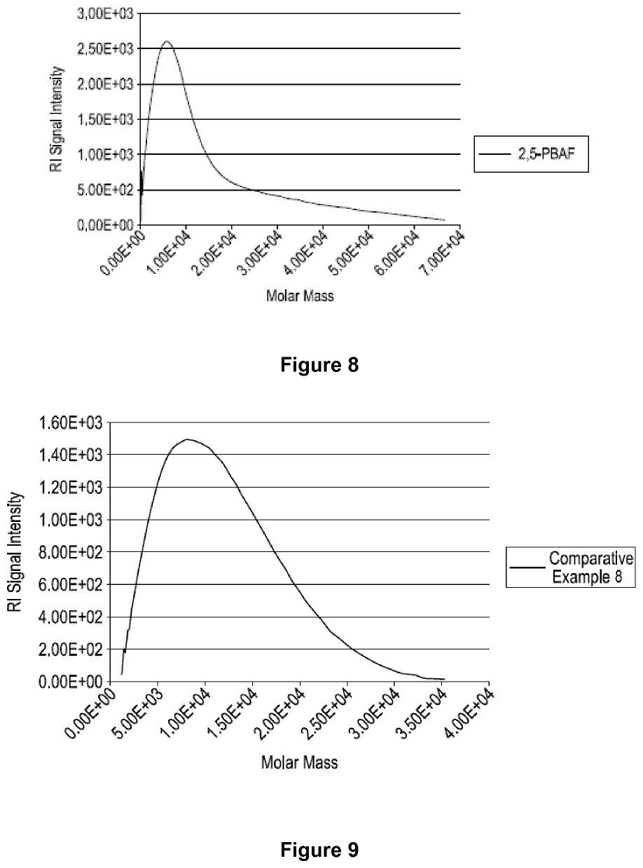

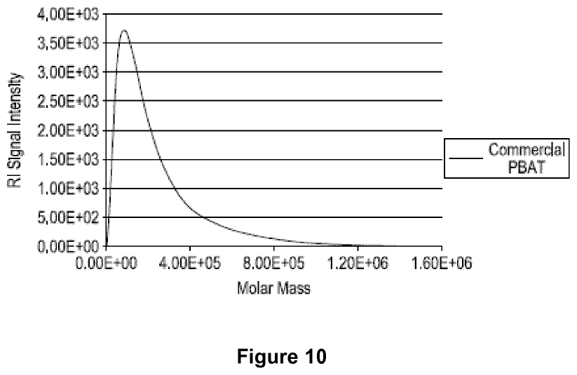

TABLE 5
GPC analysis of copolymers

| | | | | GPC |
|---|---|---|---|---|
| Copolymer | Mₙ | M_{w} | Pdᵢ | chromatogram |
| 2,5-PBAF | 862.5 | 6,121 | 7.097 | FIG. 8 |
| Comparative Example 8 | 5,582 | 8,615 | 1.544 | FIG. 9 |
| Commercial PBAT | 42,190 | 113,100 | 2.680 | FIG. 10 |

### Example 11

### Tensile Strength Measurement

Mechanical properties including tensile strength, elongation at break and Young's modulus of samples are summarised in Table 6. Film samples were prepared by heating about 8 g of copolymer in a fan-assisted oven at 160° C. for 15 min (180° C. for PBAT). The resulting films were cut into standard dumb-bell shapes (60 mm×10 mm). Film thickness was in the region of 1.5-2.0 mm. Tensile studies were conducted in triplicate using an Instron 3367 universal testing machine fitted with 1000 N capacity load cell. The initial grip separation was set at 35 mm and the crosshead speed was 20 mm/min. The results reported were the average of the three measurements (the elongation at break was obtained automatically from the software). Commercially PBAT is a typical elastomer with elongation over 293%. It has the highest tensile strength over 19.5 MPa and good Young's modulus of 100.8 MPa.

TABLE 6
Tensile strength measurement of copolymers

| | | Elongation | Young's |
|---|---|---|---|
| | Tensile strength | at break | Modulus |
| Copolymer | MPa | % | MPa |
| 2,5-PBAF | 2.2 ± 0.4 | 4.7 ± 0.8 | 75.3 ± 2.0 |
| Comparative Example 8 | 4.8 ± 0.5 | 2.3 ± 0.2 | 269.8 ± 0.2 |
| Commercial PBAT | >19.5 | >293.1 | 100.8 |

The 2,5-PBAF copolymer produced is soft like that of the commercial PBAT. The expected ratio of FDCA to adipate of about 1:1 has been incorporated into the copolymer. The observed molecular weight of 2,5-PBAF and Comparative Example 8 (PBAT) are significantly lower than that of commercial PBAT. This is expected given the relatively small scale on which the copolymerisations were conducted and will be higher in a full scale production process. The NMR data provides an indication of the relative number of constitutional repeating units (CRU) and hence an indication of molecule weight, though the GPC provides more accurate values.

The differences in the data obtained for the copolymers of the invention and the commercial BPAT may be attributed to a lack of branching in 2,5-PBAF.

### Example 12

Stabilised green waste compost is matured in a composting bin under controlled aeration conditions. Before use, the mature compost is sieved on a screen of 5 mm. The fine fraction forms the inoculum with a total solids content of approximately 50-55% and the volatile content of the total solids is more than 30%.

The standard and control materials are mixed with the inoculum in a ratio of approximately 1 to 1.5 parts of total solids to 6 parts of total solids and introduced into a reactor. These reactors are closed and put into an incubator. The temperature of the reactors is maintained at 58° C.+1-2° C. Pressurised air is pumped through a gas flow controller and blown into the composting vessel at the bottom through a porous plate. During biodegradation, solid carbon of the test sample is converted into CO₂.

The gas leaving each individual reactor is analysed at regular intervals for CO₂ and O₂ concentrations. As the flow rate is continually measured, the cumulative CO₂ production can be determined. The percentage of biodegradation is determined as the percentage of solid carbon of the test compound that is converted into CO₂.

### Example 13

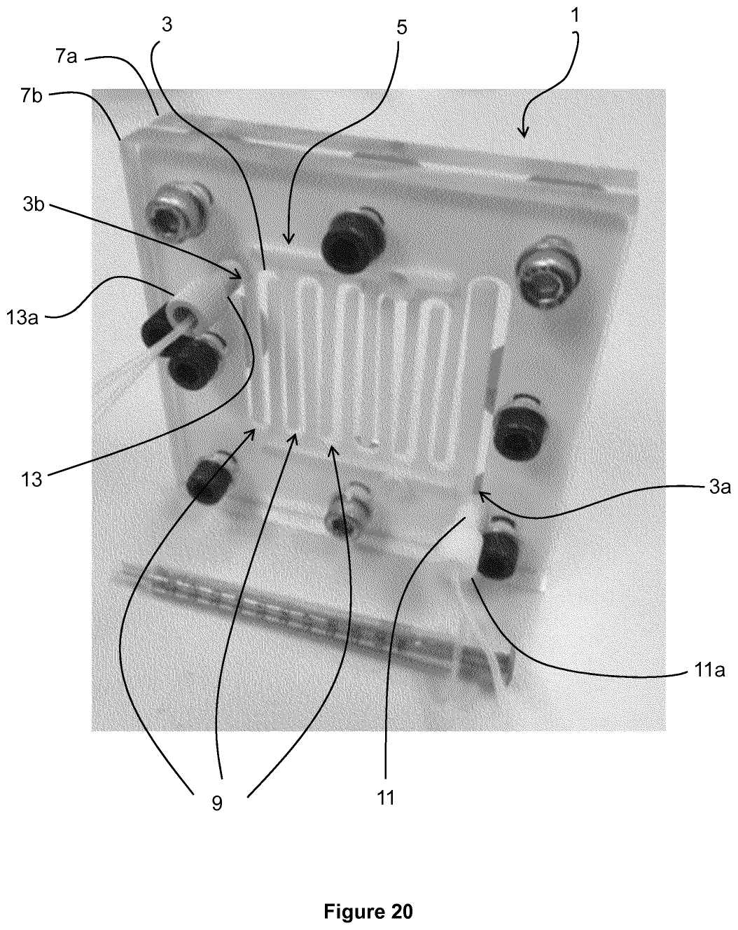

FIG. 20 depicts a perspective view of an apparatus 1 suitable for conducting a continuous flow process for oxidising a substrate (e.g. according to the present invention) on a laboratory scale.

The apparatus 1 comprises a primary tube 3 defining a primary fluid passageway (also labelled 3 herein) for flow of a fluid (such as a liquid reaction mixture). The apparatus further comprises secondary tubing 5 to provide flow of one or more further fluids to the primary fluid passageway 3.

The secondary tubing 5 is in fluid communication with the primary fluid passageway 3. In this way, one or more further fluids can be added to the primary fluid passageway 3 via the secondary tubing 5 (e.g. to permit mixture and hence reaction between components in fluid flowing through the primary passageway 3 and further fluids in the secondary tubing 5).

Apparatus 1 comprises a polytetrafluoroethylene (PTFE) primary tube 3 sandwiched between two generally square-shaped Perspex blocks 7a, 7b. The primary tube 3 comprises a tortuous primary passageway 3 having an inlet end 3a and an outlet end 3b, the passageway 3 extending in an undulating fashion between edges of the Perspex blocks 7a, 7b. The primary tube 3 comprises a total of eleven bends (collectively labelled 9) representing peaks and troughs provided at the upper and lower edges (respectively) of the Perspex blocks 7a, 7b.

The primary passageway 3 is provided with an inlet port 11 at one end for injecting fluid thereinto and an outlet port 13 for discharge at the other end. As shown, the ports 11, 13 comprise syringes 11a, 13a for input and outlet (respectively) of fluids.

The secondary tubing 5 comprises two horizontal main tubes 13 provided at the upper and lower edges of the Perspex blocks 7a, 7b, said main tubes 13 being provided with a supply of fluid thereinto (not shown). The secondary tubing further comprises a series of eleven secondary tubes (not visible) depending from one or other of the main tubes 13 (five upper, six lower). Said secondary tubes are in fluid communication with the primary passageway 3 by means of an aperture (not visible) in each secondary tube, each aperture defining an opening between each secondary tube and the primary passageway 3. Each of said eleven openings/apertures is provided at a corresponding bend 9 in the primary fluid passageway 3. Thus, the secondary tubes permit fluid communication between the main tube of the secondary tubing and the primary fluid passageway.

Such an apparatus may be useful for admitting a relatively low local concentration of one or more components (such as hydrogen peroxide) into a fluid flowing through the primary passageway, while providing a sufficient amount of that component on a bulk basis.

FIG. 25 shows a graphical representation of flow velocity through the primary fluid passageway 3, horizontal main tubes 13 and secondary tubing 5 in apparatus 1. Flow velocity was simulated with computational fluid dynamics.

For clarity, only the first (5A) and last (5F) tubes forming part of the secondary tubing 5 are labelled. Numbering of the tubes is in sequence (A-K) along the primary fluid passageway from inlet end 3a to outlet end 3b.

The pressure within tubes of the secondary tubing 5 was generally constant between different tubes, as shown in the table below.

| Secondary tube | mL/min |
|---|---|
| 5A | 0.0028 |
| 5B | 0.0019 |
| 5C | 0.0071 |
| 5D | 0.0132 |
| 5E | 0.0212 |
| 5F | 0.0323 |
| 5G | 0.0027 |
| 5H | 0.0076 |
| 5I | 0.0131 |
| 5J | 0.02 |
| 5K | 0.0295 |

The flow velocity in the primary fluid passageway 3 varies from the inlet end 3a to outlet end 3b. Approximations of the flow velocity are shown in the table below for various sections of the primary tube. Sections with similar velocity are labelled with the same letter (A-I) in the figure.

| | Velocity mms⁻¹ |
|---|---|
| A (inlet end, 3a) | 0.6 |
| B | 0.8 |
| C | 1 |
| D | 1.1 |
| E | 1.3 |
| F | 1.5 |
| G | 1.7 |
| H | 1.9 |
| I | 2.1 |

In general, velocity increases along the length of the primary fluid passageway 3 (from inlet end 3a to outlet 3b).

It will be appreciated that modifications can be made so as to balance flow rate through the apparatus 1 (e.g. to maintain a flow rate along the length of the primary fluid passageway 3). For example, the width (e.g. cross-sectional area) and/or length of tubes in the secondary tubing 5 can be configured to maintain a predetermined (e.g. constant) flow velocity along the length of the primary fluid passageway 3. Alternatively or additionally, the cross-sectional area of the primary fluid passageway 3 could similarly be configured to maintain a predetermined (e.g. constant) flow velocity along the length of the primary fluid passageway 3.

Configuration in these ways may be useful so that reagents admitted into the primary fluid passageway 3 via the secondary tubing 5 have sufficient residency time in the apparatus to undergo substantial reaction.

### Example 14

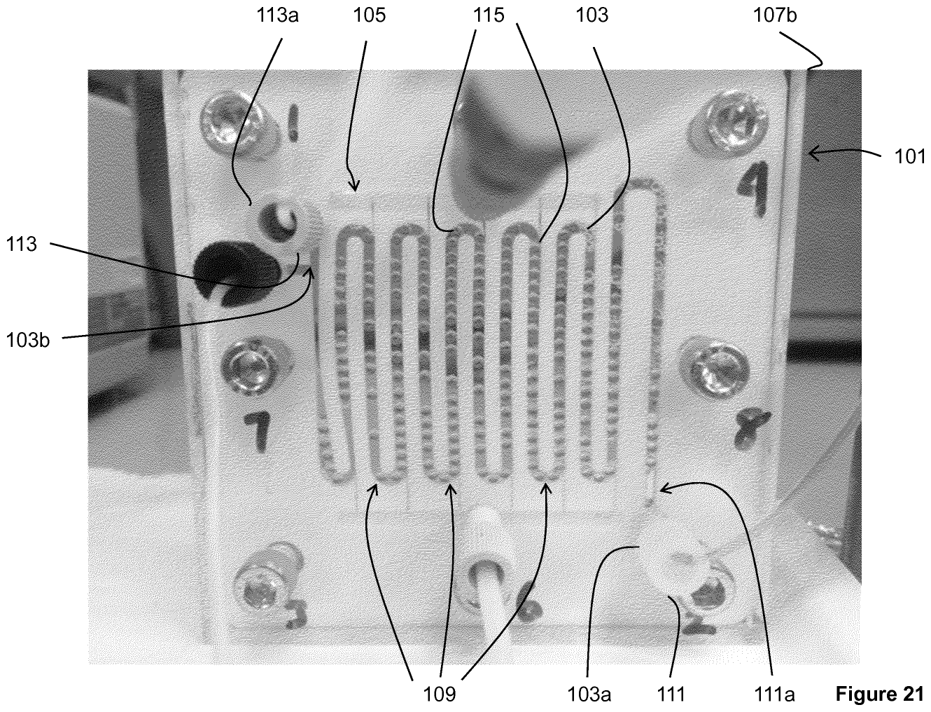

FIG. 21 depicts a front view of an apparatus 101 suitable for conducting a continuous flow process for oxidising a substrate (e.g. according to the present invention) on a laboratory scale.

The apparatus 101 is similar to the apparatus in FIG. 20 and only differences between the apparatuses will be explained below. In FIG. 21, features equivalent to features of the apparatus in FIG. 20 take the same reference number but elevated by 100.

The apparatus 101 comprises a primary tube 103 defining a primary fluid passageway (also labelled 103 herein) for flow of a fluid (such as a liquid reaction mixture). The primary passageway 103 is provided with a plurality of flow disruptors in the form of glass beads 115. The glass beads 115 disrupt the flow of liquid passing through the primary fluid passageway and thereby improve liquid mixing.

### Example 15

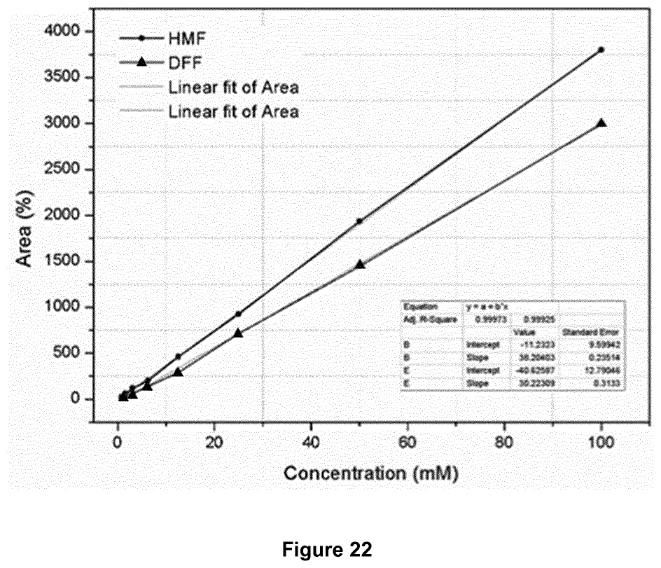

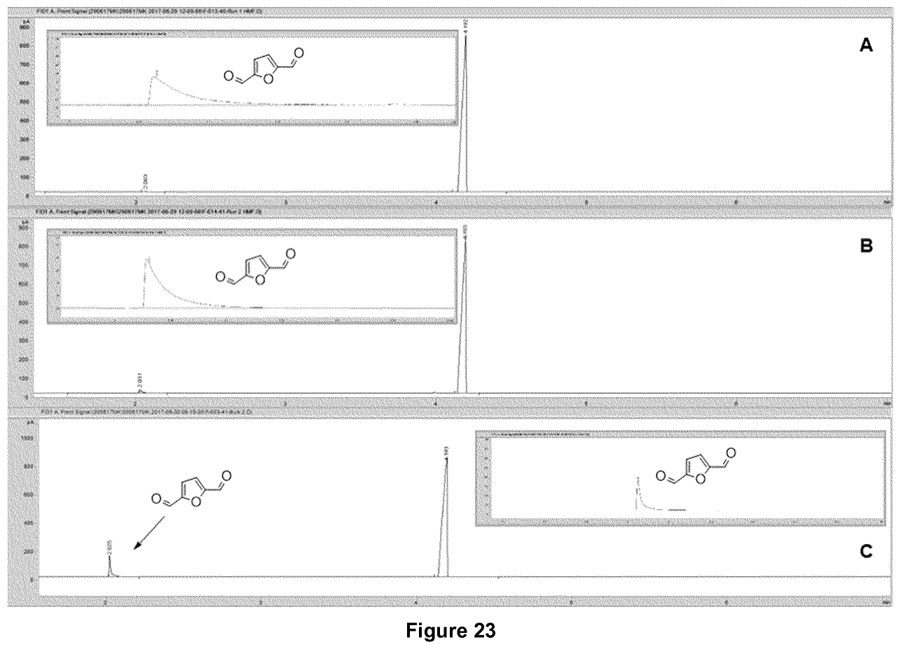

Calibration curves with an internal standard were created for quantitative gas chromatography (GC) and high-performance liquid chromatography (HPLC) analysis of hydroxymethylfurfural (HMF) and diformylfuran (DFF). The results are shown in FIG. 22. The identity of the products was additionally confirmed by GC/HPLC co-injection of reaction mixtures with chemically synthesized authentic products, or by NMR spectroscopic analysis of products obtained from reactions performed on preparative scale.

GC traces were obtained for samples of a reaction mixture during continuous-flow oxidation of 5-hydroxymethylfurfural, using GOase M₃₋₅ biocatalyst. The results are shown in FIG. 23. Traces indicate solution composition after 1 reactor volume (RV; A), 3 RVs (B) and at steady-state (C). Peak shown at 4.20 minutes=1,3,5-trimethoxybenzene as internal standard (ISTD).

### Example 16

A trickle bed reactor with 5 successive stages and 5 separate H₂O₂ side feeds was used for conversion of HMF to DFF in flow. The reactor was filled with glass beads, 2 mm diameter. Enzymes (GOase M₃₋₅ CFE, 4.80 g; CuSO₄, 0.028 g; catalase, 0.352 g; and horseradish peroxidase (HRP), 0.228 g), HMF (48 mM), and H₂O₂, 5% (w/v) (H₂O₂/HMF=50:1 mol/mol) were dissolved in potassium phosphate buffer 0.1 M and pumped through the reactor in the ratio of 1:1:1.

Reaction parameters were as follows:
Residence time=13.6 min.
Room temperature (˜21° C.)
Room pressure (˜101 kPa)
Total volume of the reactor=110 mL (22 mL per stage)

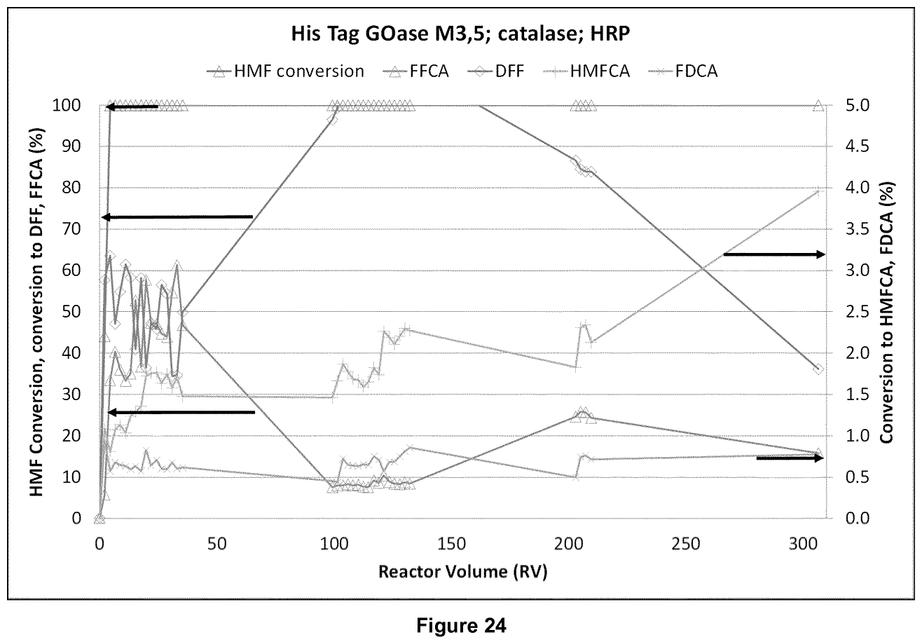

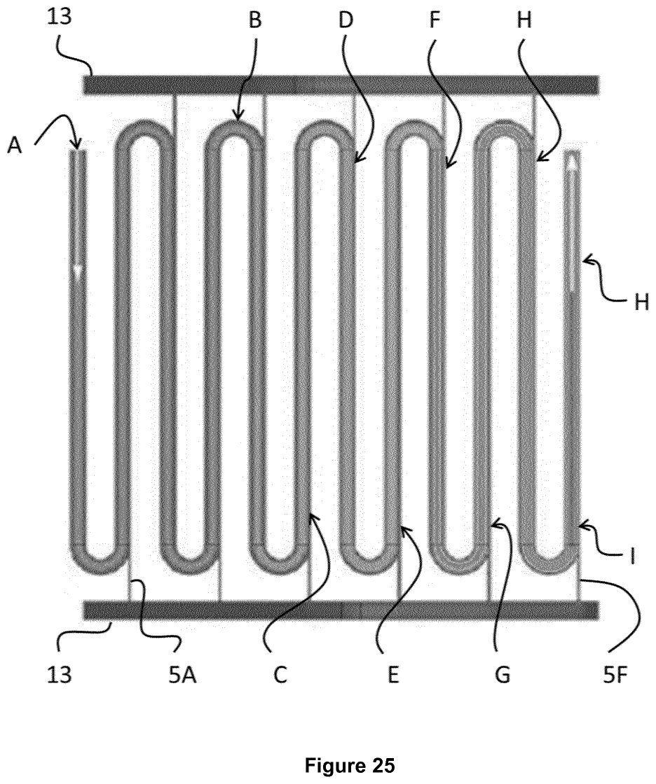

Reactor volumes were collected and analysed on HLPC. The results are shown in FIG. 24.

311 reactor volumes were collected over 3 days of continuous run, which yielded a product solution of 934 g. HMF was fully converted into DFF and HMFCA as main products of reaction. FDCA and HMFCA were also formed in smaller quantities.

The product solution of all the reactor volumes collected at 100% conversion had the following composition as measured by HPLC:
50.2% DFF
45.2% FFCA
3.6% HMFCA
1.0% FDCA

Process productivity was 1.99 g of product/L of solution/day.

### Example 17

A MultiPoint-Injection Reactor (MPIR) (See FIG. 15) was used to convert HMF to DFF in flow. This MPIR was fitted with 2H₂O₂ distributor channels, which results in 11 addition ports. Enzymes (GOase M_{3,5} CFE, 0.65 g; CuSO₄, 0.013 g; catalase, 0.013 g; and Horseradish Peroxidase (HRP), 0.010 g), HMF (30 mM), and H₂O₂ (H₂O₂/HMF at a 2:1 mol/mol) are dissolved in NaPi buffer at pH 7.4 and pumped through the reactor.

Reaction parameters are as follows:
Residence time=15 min.
Room temperature (˜21° C.)
Pressure=40 psi (276 kPa
Total volume of the reactor=2.6 mL

Reactor volumes were collected and analysed on HLPC. At steady state, 86% conversion was achieved with 100% selectivity for DFF. Process productivity was 144 g of product/L of solution/day.

Any listing or discussion of an apparently prior-published document in this specification should not necessarily be taken as an acknowledgement that the document is part of the state of the art or common general knowledge. All references disclosed herein are to be considered to be incorporated herein by reference.

Those skilled in the art will recognise or be able to ascertain using no more than routine experimentation many equivalents to the specific embodiments described herein. The scope of the present embodiments described herein is not intended to be limited to the above description, but rather is as set forth in the appended claims. Those of ordinary skill in the art will appreciate that various changes and modifications to this description may be made without departing from the spirit or scope of the present invention, as defined in the following claims.

1. A process for oxidising a substrate selected from hydroxymethylfurfural (HMF), diformylfuran (DFF), hydroxymethylfurancarboxylic acid (HMFCA) and formylfurancarboxylic acid (FFCA), said process comprising:
mixing said substrate with catalase, one or more further enzymes and hydrogen peroxide to form a reaction mixture;
wherein said one or more further enzymes have the ability to catalyse oxidation of said substrate and wherein said hydrogen peroxide is provided at a total molar ratio of at least about 0.1:1 hydrogen peroxide to substrate.

2. The process as claimed in claim 1, wherein said hydrogen peroxide is provided at a total molar ratio of at least about 1:1 hydrogen peroxide to substrate, optionally at least about 2:1 hydrogen peroxide to substrate.

3. The process as claimed in any preceding claim, wherein said hydrogen peroxide is provided at a total molar ratio of at least about 3:1 hydrogen peroxide to substrate.

4. The process as claimed in any preceding claim, wherein said hydrogen peroxide is provided at a total molar ratio of up to about 20:1 hydrogen peroxide to substrate, optionally about 15:1, optionally about 10:1 hydrogen peroxide to substrate.

5. The process as claimed in any preceding claim, wherein said mixing comprises mixing said substrate with catalase and one or more further enzymes to form a mixture, the process comprising adding hydrogen peroxide to the mixture to form the reaction mixture.

6. The process as claimed in any preceding claim, wherein said mixing comprises mixing said substrate with catalase and one or more further enzymes to form a mixture, the process comprising providing a flow of said mixture and adding hydrogen peroxide to the mixture flow to form the reaction mixture.

7. The process as claimed in claim 6, wherein said flow follows a tortuous passageway having one or more bends.

8. The process as claimed in claim 6 or 7, wherein said hydrogen peroxide is added at the bend, a subset of said bends or each bend.

9. The process as claimed in any one of claims 5 to 8, wherein said adding hydrogen peroxide comprises adding hydrogen peroxide at an amount sufficient to bring the molar ratio of the hydrogen peroxide in the reaction mixture to within about 50% of a predetermined molar ratio, optionally within about 40%, optionally within about 30%, optionally within about 20%, optionally within about 10%, optionally within about 5%, optionally within about 5% of said predetermined molar ratio, optionally approximately to said predetermined ratio.

10. The process as claimed in any one of claims 5 to 9, wherein said adding comprises pumping hydrogen peroxide.

11. The process as claimed in any one of claims 5 to 10, wherein said adding comprises adding multiple individual streams of hydrogen peroxide.

12. The process as claimed in any one of claims 5 to 11, wherein said adding comprises adding at least 5 individual streams of hydrogen peroxide, optionally at least 7, optionally at least 10, optionally at least 11 individual streams of said hydrogen peroxide.

13. The process as claimed in any one of claims 5 to 12, wherein said adding comprises continuously adding or periodic adding.

14. The process as claimed in claim 13, wherein said periodic adding comprises adding 1, 2, 3, 4, 5, 6, 7, 8, 9, or 10 (or more) portions of said hydrogen peroxide.

15. The process as claimed in claim 13 or 14, wherein said periodic adding comprises adding two or more portions of hydrogen peroxide, the addition of each portion being separated by a time interval of from about 1 second to about 60 minutes, optionally about 5 minutes to about 25 minutes, optionally about 10 minutes to about 20 minutes, optionally about 15 minutes.

16. The process as claimed in any one of claims 6 to 15, wherein said flow has a velocity and wherein the process is configured to maintain a predetermined flow velocity along a path of the flow.

17. The process as claimed in claim 16, wherein said provision of hydrogen peroxide is configured to maintain the predetermined flow velocity.

18. The process as claimed in any preceding claim, wherein said one or more further enzymes comprise metal.

19. The process as claimed in any preceding claim, wherein said one or more further enzymes comprise iron, molybdenum and/or copper metal.

20. The process as claimed in any preceding claim, wherein the substrate is selected from 2,5-diformylfuran (DFF), 5-hydroxymethyl-2-furancarboxylic acid (HMFCA) and/or 5-formylfuran-2-carboxylic acid (FFCA).

21. The process as claimed in any preceding claim, wherein the process for the formation of furandicarboxylic acid, optionally 2,5-furandicarboxylic acid (2,5-FDCA).

22. The process as claimed in any preceding claim, wherein said one or more further enzymes comprise xanthine oxidoreductase (XOR), aldehyde oxidase, aldehyde dehydrogenase (ALD), alcohol oxidase, galactose oxidase variant (such as galactose oxidase variant M₃₋₅, GOase M₃₋₅), ketoreductase (KRED) and/or nicotinamide oxidase (NOX).

23. The process as claimed in claim 22, wherein said xanthine oxidoreductase is selected from E. coli XDH, Rhodococcus capsulatus xanthine dehydrogenase (XDH) single variant E232V, and double mutant XDH E232 V/R310, and periplasmic aldehyde oxidase (PaoABC).

24. The process as claimed in claim 22 or 23, wherein the xanthine oxidoreductase is periplasmic aldehyde oxidase (PaoABC).

25. The process as claimed in any preceding claim, wherein said one or more further enzymes comprise galactose oxidase variant M₃₋₅ (GOase M₃₋₅).

26. The process as claimed in any preceding claim, wherein said one or more further enzymes comprise (a) galactose oxidase variant (such as galactose oxidase variant M₃₋₅ (GOase M₃₋₅)] and/or ketoreductase (KRED); and (b) one or more of xanthine oxidoreductase (XOR), aldehyde dehydrogenase (ALD) and nicotinamide oxidase (NOX).

27. The process as claimed in any preceding claim, wherein said catalase is immobilised.

28. The process as claimed in any preceding claim, wherein said one or more further enzymes comprise one or more immobilised enzymes.

29. The process as claimed in claim 28, wherein said one or more further enzymes comprise immobilised periplasmic aldehyde oxidase (PaoABC).

30. The process as claimed in claim 28 or 29, wherein said one or more further enzymes comprise immobilised galactose oxidase variant [such as galactose oxidase variant M₃₋₅ (GOase M₃₋₅)].

31. The process as claimed in any preceding claim, wherein said mixing further comprises mixing with horseradish peroxidase (HRP) and/or metal complex.

32. The process as claimed in any preceding claim, wherein said mixing further comprises mixing with nicotinamide adenine dinucleotide phosphate (NADP⁺) and/or nicotinamide adenine dinucleotide (NAD⁺).

33. The process as claimed in any preceding claim, wherein said one or more further enzymes comprises nicotinamide oxidase (NOX) and wherein said mixing further comprises mixing with nicotinamide adenine dinucleotide phosphate (NADP⁺) and/or nicotinamide adenine dinucleotide (NAD⁺).

34. The process as claimed in any preceding claim, wherein one or more further enzymes comprises ketoreductase (KRED) and wherein said mixing comprises mixing with nicotinamide adenine dinucleotide phosphate (NADP⁺) and/or nicotinamide adenine dinucleotide (NAD⁺).

35. The process as claimed in any preceding claim, wherein said diformylfuran (DFF), hydroxymethylfurancarboxylic acid (HMFCA) and/or formylfurancarboxylic acid (FFCA) is/are obtained by oxidation of hydroxymethylfurfural (HMF).

36. The process as claimed in any preceding claim, wherein said hydroxymethylfurfural (HMF) is obtained from glucose and/or fructose.

37. The process as claimed in claim 36, wherein said glucose and/or fructose is obtained from cellulose.

38. The process as claimed in claim 37, wherein said cellulose is obtained from lignocellulose.

39. An apparatus for a flow process of oxidising a substrate, said apparatus comprising:
a primary tube defining a primary fluid passageway for flow of a first fluid;
secondary tubing defining a secondary passageway for adding one or more further fluids to the primary fluid passageway, said secondary tubing having one or more apertures to permit fluid communication of the secondary passageway with the primary passageway.

40. The apparatus according to claim 39, wherein the first fluid is liquid.

41. The apparatus according to claim 39 or 40, wherein the total cross-sectional area of the aperture(s) of the secondary tubing is about 10 to 30 times smaller than the cross-sectional area of the primary passageway, optionally about 15 to 25 times smaller, optionally about 20 times smaller than the cross-sectional area of the primary passageway.

42. The apparatus as claimed in any one of claims 39 to 41, wherein said apparatus is configured to control flow of fluid through said primary passageway and/or flow of fluid through said secondary passageway based on a molar ratio of components in the first fluid of the primary passageway.

43. The apparatus according to any one of claims 39 to 42, wherein the primary tube is configured to provide a tortuous passageway, having one or more bends.

44. The apparatus as claimed in claim 43, wherein the or each aperture of the secondary tubing is provided at the bend, a subset of said bends or each bend in the primary passageway.

45. The apparatus according to any one of claims 39 to 44, wherein the primary tube is provided with one or more flow disruptors.

46. The apparatus according to any one of claims 39 to 45, wherein the flow disruptor(s) are particles, such as inert particles, optionally glass beads.

47. The apparatus according to any one of claims 39 to 46, wherein the flow disruptor(s) may be sized to closely fit inside the primary tube.

48. The apparatus as claimed in any one of claims 39 to 47, wherein said secondary tubing comprises one or more main secondary tubes, the or each main secondary tube defining an individual fluid passageway and being in fluid communication with a series of further subordinate secondary tubes, said secondary tubes each having the said apertures for fluid communication with the primary passageway, said further subordinate secondary tubes fluidly connecting the primary passageway and the fluid passageway of the main secondary tube.

49. The apparatus as claimed in any one of claims 39 to 48, wherein said secondary tubing comprises at least 5 individual fluid passageways, optionally at least 7, optionally at least 10, optionally at least 11 individual fluid passageways.

50. The apparatus as claimed in any one of claims 39 to 49, wherein a cross-sectional area and/or length of tubes in the secondary tubing may be configured to maintain a predetermined flow velocity along the length of the primary tube.

51. The apparatus as claimed in any one of claims 39 to 50, wherein a cross-sectional area of the primary tube may be configured to maintain a predetermined flow velocity along the length of the primary tube.

52. A process for the formation of a mono- or diester of furandicarboxylic acid from furandicarboxylic acid, the process comprising mixing furandicarboxylic acid, an alcohol and a catalyst, wherein the furandicarboxylic acid is obtained by a process as defined in any one of claims 1 to 38.

53. The process as claimed in claim 52, wherein said alcohol is selected from methanol and ethanol.

54. The process as claimed in claim 52 or 53, wherein the catalyst is an organic acid or inorganic acid.

55. The process as claimed in any one of claims 52 to 54, wherein the catalyst is sulphuric acid.

56. A process for the formation of a copolymer comprising the copolyester of:
(a) furandicarboxylic acid (FDCA) and/or a mono- or diester of furandicarboxylic acid; and
(b) at least one diol;
wherein the process comprises polymerising components (a) and (b) wherein the furandicarboxylic acid is obtained by a process as defined in any one of claims 1 to 38, and/or wherein the mono- or diester of furandicarboxylic acid is obtained by a process as defined in any one of claims 52 to 55.

57. The process as claimed in claim 56, wherein the furandicarboxylic acid, or mono- or diester of furandicarboxylic acid is selected from 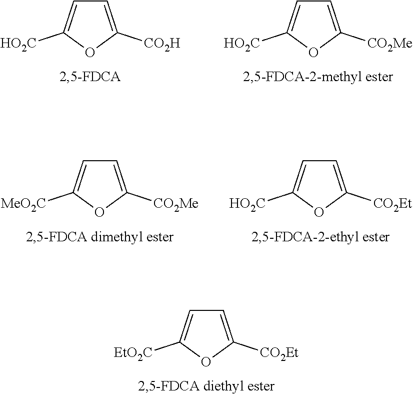
and a combination thereof.

58. The process as claimed in claim 56 or 57, wherein the mono- or diester of furandicarboxylic acid is selected from 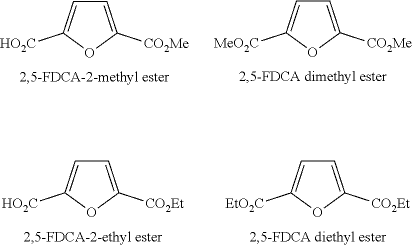
and a combination thereof.

59. The process as claimed in any one of claims 56 to 58, comprising said at least one mono- or diester of furandicarboxylic acid.

60. The process as claimed in any one of claims 56 to 59, wherein said at least one diol comprises an aliphatic diol.

61. The process as claimed in any one of claims 56 to 60, wherein the at least one diol comprises: 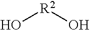
wherein R² is a straight, branched or cyclic C₂ to C₁₀ alkylene.

62. The process as claimed in any one of claims 56 to 61, wherein the at least one diol comprises a diol selected from 1,2-ethanediol, 1,4-butanediol, and a combination thereof.

63. The process as claimed in any one of claims 56 to 62, wherein the copolymer comprises the copolyester of:
(a) at least one furandicarboxylic acid (FDCA) or a mono- or diester of furandicarboxylic acid;
(b) at least one diol; and
(c) at least one dicarboxylic acid or a mono- or diester derivative thereof.

64. The process as claimed in claim 63, wherein said at least one dicarboxylic acid, or mono- or diester derivative thereof acid, comprises an aliphatic, heteroaromatic and/or aromatic dicarboxylic acid, or mono- or diester derivative thereof.

65. The process as claimed in claim 63 or 64, wherein said at least one dicarboxylic acid, or mono- or diester derivative thereof acid, comprises an aliphatic dicarboxylic acid, or mono- or diester derivative thereof.

66. The process as claimed in claim 65, wherein the aliphatic dicarboxylic acid, or mono- or diester derivative thereof, is: 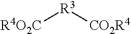
wherein R³ is a straight, branched or cyclic, C₁ saturated or C₂ to C₁₀ saturated or unsaturated alkylene, and wherein each R⁴ independently represents H or a straight, branched or cyclic, C₁ to C₈ (optionally C₁ to C₆) alkyl group.

67. The process as claimed in claim 65 or 66, wherein the aliphatic dicarboxylic acid, or mono- or diester derivative thereof, is selected from adipic acid, adipic acid monomethyl ester, adipic acid dimethyl ester, adipic acid monoethyl ester, adipic acid diethyl ester, succinic acid, succinic acid monomethyl ester, succinic acid dimethyl ester, succinic acid monoethyl ester, succinic acid diethyl ester, and a combination thereof.

68. The process as claimed in any one of claims 64 to 67, wherein said at least one dicarboxylic acid, or mono- or diester derivative thereof, comprises an aromatic dicarboxylic acid, or mono- or diester derivative thereof.

69. The process as claimed in any one of claims 56 to 68, wherein the copolymer comprises the copolyester of:
(A) the dimethyl ester or diethyl ester of furandicarboxylic acid (FDCA) (optionally 2,5-furandicarboxylic acid (2,5-FDCA));
(B) 1,4-butanediol; and
(C) dimethyl ester or diethyl ester of adipic acid.

70. The process as claimed in any one of claims 56 to 69, comprising the diethyl ester of furandicarboxylic acid.

71. The process as claimed in any one of claims 56 to 70, wherein the copolymer comprises the copolyester of (a) from 1 to 98 mol % of at least one 2,5-furandicarboxylic acid or a mono- or diester of 2,5-furandicarboxylic acid (2,5-FDCA);
(b) from 1 to 98 mol % of at least one diol; and
(c) when present, from 1 to 98 mol % of at least one aliphatic dicarboxylic acid or a mono- or diester derivative thereof.

72. A copolymer obtainable by a process as defined in any one of claims 56 to 71.

73. A process or copolymer substantially as hereinbefore described, with reference to the figures.
